(19) United States
(12) Patent Application Publication
(10) Pub. No.: US 2007/0088247 A1
(43) Pub. Date: Apr. 19, 2007

(54) Apparatus and method for thermal ablation of uterine fibroids

(75) Inventors: Mordechai Bliweis, Haifa (IL); Nir Berzak, Zikhron-Yaakov (IL); Shimon Livneh, Kiryat-Tivon (IL); Yaron Hefetz, Herzlia (IL); Yaron Tal, Tel-Mond (IL); Roni Zvuloni, Haifa (IL)

Correspondence Address: Martin D. Moynihan;PRTSI, Inc., P.O. Box 16446, Arlington, VA, 22215, US

(73) Assignee: Galil Medical Ltd., Yokneam (IL)

(21) Appl. No.: 11/637,095

(22) Filed: Dec. 12, 2006

Related U.S. Application Data
(63) Continuation-in-part of application No. 11/055,597, filed on Feb. 11, 2005.
Application No. 11/055,597 is a continuation of application No. 09/987,689, filed on Nov. 15, 2001, now abandoned.
Application No. 09/987,689 is a continuation-in-part of application No. 09/860,486, filed on May 21, 2001, now Pat. No. 6,706,037.
(63) Continuation-in-part of application No. 11/185,699, filed on Jul. 21, 2005.
Application No. 11/185,699 is a division of application No. 10/151,310, filed on May 21, 2002, now abandoned.
(60) Provisional application No. 60/242,455, filed on Oct. 24, 2000.
(60) Provisional application No. 60/300,097, filed on Jun. 25, 2001.
(60) Provisional application No. 60/291,990, filed on May 21, 2001.
(60) Provisional application No. 60/762,110, filed on Jan. 26, 2006.
(60) Provisional application No. 60/750,833, filed on Dec. 16, 2005.

(51) Int. Cl. A61B 18/18; A61B 17/20

(52) U.S. Cl. 604/22; 606/21

(57) ABSTRACT

The present invention relates to apparatus and methods for thermally ablating uterine fibroids. More particularly, the present invention relates to a conduit having a plurality of channels for delivering a plurality of thermal ablation probes to an organic target such as a uterine fibroid, the probes being delivered in such configuration and orientation as to enable efficient and thorough ablation of the fibroid. In a preferred embodiment, the conduit is formed as a sleeve having a large central lumen sized to accommodate a hysteroscope, channels sized to accommodate cryoprobes are used as thermal ablation probes, and comprises thermal insulation materials serving to protect the cervix from damage by cold. The present invention further relates to bent cryoprobes usable in conjunction with such a conduit and designed to exit therefrom in a desired configuration useful for ablating a large fibroid.

### RELATED APPLICATIONS

This Application is a continuation-in-part of pending U.S. patent application Ser. No. 11/055,597 filed Feb. 11, 2005, which is a continuation of U.S. patent application Ser. No. 09/987,689 filed Nov. 15, 2001, now abandoned, which is a continuation-in-part of U.S. patent application Ser. No. 09/860,486 filed May 21, 2001, now U.S. Pat. No. 6,706,037 issued Mar. 16, 2004, which claims the benefit of U.S. Provisional Patent Application No. 60/242,455, filed Oct. 24, 2000.

This Application is also a continuation-in-part of pending U.S. patent application Ser. No. 11/185,699 filed Jul. 21, 2005, which is a divisional of U.S. patent application Ser. No. 10/151,310 filed May 21, 2002, now abandoned, which claims the benefit of U.S. Provisional Patent Application No. 60/300,097 filed Jun. 25, 2001 and U.S. Provisional Patent Application No. 60/291,990 filed May 21, 2001.

This Application also claims the benefit of U.S. Provisional Patent Application No. 60/762,110 filed Jan. 26, 2006.

This Application further claims the benefit of U.S. Provisional Patent Application No. 60/750,833 filed Dec. 16, 2005.

The contents of the above Applications are incorporated herein by reference.

### FIELD AND BACKGROUND OF THE INVENTION

The present invention relates to apparatus and methods for thermal ablation of a surgical target within a body of a patient. More particularly, the present invention relates to use of a conduit having a plurality of channels for delivering a plurality of thermal ablation probes to an organic target such as a uterine fibroid, the probes being delivered in such configuration and orientation as to enable efficient and thorough ablation of the target. In a preferred embodiment, the conduit is formed as a sleeve having a large central lumen sized to accommodate an optical hysteroscope and a plurality of channels sized to accommodate thermal ablation probes such as cryoprobes, the sleeve further comprising thermal insulation materials serving to protect tissues distant from the target from thermal damage. The present invention further relates to bent cryoprobes usable in conjunction with such a conduit and designed to exit therefrom in a desired configuration useful for ablating a large target, and further relates to thermal probes having low-profile connectors which facilitate independent movement of probes and sleeve when probes inserted through the sleeve are inserted into the target. The invention is particularly useful for facilitating cryoablation of uterine fibroids.

Uterine fibroids are benign muscular hyperplasia of the muscular wall of the uterus. Methods currently used for treating fibroids include hysterectomy, trans-vaginal resectoscopy using a hysteroscope in conjunction with a wire loop electrode, and cryoablation using an endoscope for manipulating flexible cryoprobes. Endoscopic cryoablation is currently limited to treating fibroid growth on outer walls of the uterus. Resectoscopes (“electric snares”) serve to treat fibroids penetrating into the uterus. However, often, when a large part of a fibroid is contained within the muscular walls rather than simply projecting into the uterus, repeated treatments are required.

Thus, there is a widely recognized need for, and it would be highly advantageous to have, an apparatus and method enabling effective, rapid, convenient and thorough treatment of uterine fibroids positioned within a uterus or embedded in a uterine wall.

Prior art relevant to the flexibility of cryoprobes and of equipment useable with cryoprobes includes U.S. Pat. No. 5,978,697 for “System and method for MRI-guided cryosurgery” to Maytal et al., which discloses a flexible gas line for a cryoablation probe.

U.S. Pat. No. 6,936,045 for “Malleable cryosurgical probe” to Yu et al. is similarly relevant. Yu discloses a malleable cryosurgical probe which includes a cryostat assembly and a cryoprobe assembly, wherein the cryostat assembly includes an elongated shaft assembly having at least one malleable segment and a closed distal end, and the shaft assembly includes at least one freezing portion, at least one thermally insulated portion, and a thermally insulating element positioned about the thermally insulated portion.

U.S. Patent Application No. 20020188287, entitled “Apparatus and method for cryosurgery within a body cavity”; by Roni Zvuloni et al. discloses a system for facilitating delivery of cryoprobes to cryoablation targets within the body. Zvuloni discloses an apparatus and method for cryosurgery within a body cavity, which apparatus comprises a trocar installable in an external passageway opened in a wall of a body cavity of a patient, the trocar having a portal serving to maintain and control the external passageway after installation of the trocar, the portal being useable for transmitting therethrough at least one surgical instrument for use during a surgical procedure. The apparatus further includes at least one cryoprobe deployable through the portal of the trocar into a body cavity. The cryoprobe is operable to be positioned in the body cavity in a selected orientation and position, and to cryoablate a tissue within the body cavity when in that selected orientation and position.

U.S. Patent Application No. 2004/0143252 entitled “Echogenic needle for transvaginal ultrasound directed reduction of uterine fibroids and an associated method” by Bradley Shawn Hurst discloses a vaginal ultrasonic probe with a guide for a thermal or RF treatment needle comprising an echogenic surface. Hurst discloses a method of guiding the treatment needle to the fibroid using trans-vaginal ultrasound imaging, aided by enhanced ultrasonic visibility of the echogenic needle. The disclosed system does not provide visual inspection of the treated location and discloses only straight, rigid treating needles.

Rigid and flexible hysteroscopes useful for inspection and diagnosis within the uterus are well known in the art. Commercially available rigid hysteroscope may be found, for example, at http://www.conmed.com/products-endoscopy-scope.php.

Flexible hysteroscopes are also commercially available. Olympus Surgical & Industrial America Inc., for example, sells a “model HYF-V” flexible fiber-optic hysteroscope.

The maximum practical diameter of hysteroscopes is limited by the size of the cervix opening. The outer diameter of hysteroscopes and resectoscopes is generally limited to about 9 mm, that being a typical maximum size achievable by mechanical non-destructive dilation of the cervix.

The hysteroscopes known to prior art, the maximum size of working channels provided by hysteroscopes (“working channels” being channels enabling introduction of tools additional to the optical components of the hysteroscope into a treatment area) is consequently also limited. Some commercially available hysteroscopes have working channels with 2 mm diameters. Some available hysteroscopes have an outer sleeve with working channel for insertion of tools, for example electrodes. These sleeves have tubular construction and fit around a thin hysteroscope. An example is the “Gynecare Versapoint” sold by Gynecare Inc. of Somerville, N.J.

Thermal treatment probes with diameters of less then 2 mm, having sharp points on a short distal tip and also having a flexible hose for providing high-pressure gas, are available from Galil Medical Ltd. of Yokneam, Israel, and are used in endoscopic operations.

B-K Medical (http://www.bkmed.com/applications/surgery/intraoperative.asp) sells an ultrasound transducer usable for guiding RF ablation probes to general ablation targets and which enables withdrawing the ultrasound transducer while leaving the ablation probes in place. Uterine surgery, however, provides unique access limitations, in consequence of which hysteroscopes and resectoscopes currently in use for uterine treatment enable only one thermal ablation probe to be delivered to a treatment target such as a fibroid. Thus in the case of cryoablation for example, only a single cryoprobe can be delivered to a fibroid according to technologies known to prior art. However, some fibroids are larger than the maximum size of the ice ball which may be produced by a small-diameter cryoprobe. A cryosurgical ablation cycle may require up to thirty minutes to complete, and the treated tissue must be at least partially thawed to enable an inserted and cooled cryoprobe to be subsequently displaced to a second location.

Thus it would be advantageous to be able to deliver a plurality of cryoprobes to a large fibroid, and there perform cryoablation using a plurality of cryoablation probes concurrently.

Thus, there is a widely recognized need for, and it would be highly advantageous to have, an apparatus capable of passing easily through a dilated cervix, yet operable to deliver a plurality of treatment probes to a fibroid. It would further be highly advantageous to have an apparatus capable of presenting such a delivered plurality of cryoprobes in a configuration appropriate for treating a large fibroid whose external dimensions largely exceed the diameter of a dilated cervix.

It is an additional limitation of currently known technologies, wherein a working channel of a therapeutic hysteroscope is used to introduce a thermal probe into a treatment target, that external connections to the thermal probe and internal dimensions of the working channel are such that probe and hysteroscope are necessarily paired one within the other for the duration of the treatment phase of the thermal operation, thereby preventing use of such a therapeutic hysteroscope with more than one such treatment probe at a time. Since ablation targets (such as fibroids) are often too large to be treated by a single therapeutic operation of a single probe (e.g., by a single cooling cycle of a single cryoprobe), there is a widely recognized need for, and it would be highly advantageous to have, a probe/hysteroscope arrangement whereby a probe passing through a working channel of a hysteroscope or other delivery apparatus might be inserted into a treatment target, then liberated from that working channel prior to full or partial therapeutic activation of the probe, thereby enabling delivery of additional therapeutic probes or other surgical tools through that working channel, the plurality of treatment tools thus delivered sequentially through a single working channel may yet be positioned in or near a common target and be operated simultaneously when so positioned.

It is an additional limitation of currently known technologies that the form of working channels of known hysteroscopes and the form of known thermal treatment probes used therein limit the maneuverability of those probes in the uterine context. It is a further limitation of known technologies that cold induced by the shafts and cryogen input and exhaust lines of croprobes used for intrauterine cryoablation tends to endanger tissues of the cervix. Thus, there is a widely recognized need for, and it would be highly advantageous to have, an apparatus enabling to overcome these limitations.

### SUMMARY OF THE INVENTION

According to one aspect of the present invention there is provided a multiprobe delivery system with slit. A system for delivering a treatment probe to a treatment target within a body cavity, comprising a delivery sleeve having a distal portion operable to be inserted into a body cavity, the delivery sleeve being sized to accommodate at least one treatment probe and being operable to deliver a treatment head of the treatment probe to a vicinity of the treatment target when the distal portion of the sleeve is inserted in the body cavity, the delivery sleeve being further characterized in that the sleeve comprises an opening running along its length, the opening being sized to permit passage therethrough of the treatment probe.

According to further features in preferred embodiments of the invention described below, the system further comprises a treatment probe lumen sized to accommodate the at least one treatment probe, the lumen being switchable between an open state permitting a treatment probe to enter and exit the treatment probe lumen and a closed state which prevents treatment probes from entering and from exiting the treatment probe lumen. Preferably the open state is characterized by a first configuration wherein the opening is aligned with the treatment probe lumen, enabling translation of a treatment probe into and out of the lumen, and the closed state is characterized by a second configuration wherein the opening is unaligned with the treatment probe lumen and translation of a treatment probe into and out of the treatment probe lumen is prevented. Transition from the open state to the closed state may be effected by rotating a cover of the delivery sleeve with respect to a body of the delivery sleeve.

According to further features in preferred embodiments of the invention described below, the delivery sleeve is sized to accommodate both the at least one a treatment probe and also a visual guiding apparatus, and the treatment probe is prevented from passage through the opening when the probe and the visual guiding apparatus are both inserted in the sleeve, and the treatment probe is enabled to pass through the opening when the probe is inserted in the sleeve and the visual guiding apparatus is not inserted therein. The visual guiding apparatus is an optical hysteroscope. The system preferably comprises a plurality of treatment probes.

According to still further features in preferred embodiments of the invention described below, the distal portion of the delivery sleeve is so shaped and dimensioned as to be capable of insertion into a uterine lumen through a cervix, the treatment probe is a cryoprobe, and the delivery sleeve comprises an echogenic surface. Preferably, at least a portion of the delivery sleeve comprises heat-insulating material. Second and third sleeves comprising heat-insulating material may also be provided.

According to another aspect of the present invention there is provided a multiple-probe delivery system having multiple channels each sized to accommodate a treatment probe. A system for delivering a plurality of thermal treatment probes to a treatment target within a body cavity, comprises a sleeve having a first lumen sized to accommodate an optical hysteroscope, and a plurality of working channels each sized to accommodate a treatment probe. The first lumen may be positioned centrally within the sleeve, and the working channels are positioned circumferentially around the central lumen, or the working channels may be positioned asymmetrically with respect to the first lumen. The system preferably comprises a plurality of treatment probes each sized to be insertable within at least one of the working channels.

According to further features in preferred embodiments of the invention described below, at least one of the treatment probes comprises a proximal connector operable to connect the at least one probe to a cryogen source, the connector being of a diameter not substantially greater than a diameter of the probe. The treatment probe may be a cryoprobe, and may be a pre-bent treatment probe operable to be inserted into a straight channel and to assume a bent configuration when exiting a distal end of that channel. In a preferred embodiment the system comprises a plurality of pre-bent treatment probes disposable within the plurality of working channels in such orientation that when the pre-bent treatment probes extend from a distal end of the sleeve, a distance of one of the treatment heads from at least one other of the treatment heads is greater than a diameter of the sleeve. In an alternative preferred embodiment, distal ends of at least some of the plurality of channels diverge as they approach a distal end of the sleeve.

According to yet another aspect of the present invention there is provided a system for delivering a thermal treatment probe to a treatment target within a body cavity, comprising a sleeve having a first lumen sized to accommodate a visual guiding apparatus, a working channel sized to accommodate a treatment probe, and a treatment probe which comprises a proximal connector operable to connect the probe to a cryogen source, the connector being of a diameter not substantially greater than a diameter of the probe. The treatment probe may be a cryoprobe. The treatment probe may also be a pre-bent probe.

According to still another aspect of the present invention there is provided a probe sized and shaped to traverse a working channel of an endoscope, comprising a shaft having a maximum diameter D sized and shaped to enable passage through a working channel of an endoscope, a treatment head positioned at a distal portion of the shaft and operable to treat a target tissue within a body cavity when supplied with a material substance transported to the treatment head through the shaft, and a connector positioned at a proximal portion of the shaft and operable to connect the shaft to a source of the material substance, the connector having a diameter not superior to the diameter D of the shaft. Optionally, the treatment head comprises a member operable to be anchored to the target tissue. Preferably, the treatment head is operable to be cooled when a cryogen is supplied through the connector.

According to a further aspect of the present invention there is provided an insulating device for protecting a cervix during thermal cryoablation within a uterus, comprising a distal portion formed and dimensioned so as to be operable to non-destructively penetrate a cervix and a lumen sized to accommodate cryogen supply and exhaust lines of at least one cryoprobe. The device comprises heat-insulating materials operable to at least partially protect tissues of a cervix from thermal damage when the device is positioned within the cervix and a cryoprobe having cryogen supply and exhaust lines passing within the lumen is used to thermally ablate tissues within the uterus. The device preferably comprises a proximal portion sufficiently broad to prevent penetration of the proximal portion through the cervix and a bulge in a region of the distal portion of the device, the bulge being so positioned as to impede withdrawal of the device from the cervix once the distal portion of the device is inserted into the cervix. In a preferred embodiment the device further comprises a longitudinal slit enabling to push a cryogen supply line into the lumen of the device from a position alongside the device. Alternatively, the lumen may be openable and closeable, opening of the device enabling lateral introduction of an adjacent cryogen supply line into the lumen, and closing of the device enabling to protect cervical tissues when the device is inserted in the cervix and a cryogen supply line introduced into the lumen is cooled.

According to another aspect of the present invention there is provided a probe delivery system which delivers probes which spread out as they extend beyond a distal end of the delivery system, thereby enabling to treat a large extended treatment target. A system for treating target tissue within a body cavity comprises a plurality of treatment tools, each comprises a distal portion formed as a treatment head operable to treat the target tissue, and a sleeve having a distal end insertable into a body cavity and operable to deliver the treatment heads of the plurality of treatment tools to a vicinity of the target tissue within the body cavity. The sleeve and the tools are so formed and configured that if the distal portion of the sleeve is inserted in the body cavity and the plurality of tools is delivered through the sleeve to the vicinity of the target tissue, the treatment heads of the plurality of tools are constrained to exit the sleeve in a dispersed configuration such that a distance between one of the treatment heads and at least one other of the treatment heads is greater than a diameter of the sleeve. In a preferred embodiment at least one of the treatment tools is a thermal ablation tool such as a cryoprobe. In a preferred embodiment the sleeve comprises a plurality of channels each sized to accommodate a treatment tool, distal portions of the plurality of channels being so shaped and positioned that average distance of the channels one from another increases as the channels approach a distal end of the sleeve. In a further preferred embodiment the sleeve comprises a channel and at least one of the treatment tools is a pre-bent tool operable to be inserted into the channel and to be advanced therethrough, the pre-bent tool being further operable to resume a bent configuration when advanced beyond a distal end of the channel. In yet another preferred embodiment the sleeve comprises a channel having a proximal portion, a distal portion, and a pivot joining the proximal and the distal portions, the pivot enabling to change angle of orientation of the distal portion with respect to the proximal portion. A distal portion of a one of the treatment tools is operable to emerge from the distal portion of the channel in a direction influenced by the angle of orientation of the distal portion with respect to the proximal portion of the channel. The system preferably further comprises a visual guiding apparatus and the sleeve further comprises a lumen sized to accommodate a visual guiding apparatus such as an optical hysteroscope.

According to still another aspect of the present invention there is provided a sleeve for delivering a plurality of treatment probes to a surgical target within a body cavity, the sleeve comprises a plurality of working channels each channel sized to accommodate a treatment probe, at least some of the channels diverge at a distal portion of the sleeve.

According to a further aspect of the present invention there is provided a treatment probe for treating an organic target within a body, which probe is sufficiently flexible to assume a straight configuration when so constrained by introduction into a straight channel, yet which assumes a bent configuration when freed of the constraint. In a preferred embodiment, the treatment probe is a cryoprobe. The probe may further comprise a marking showing information describing the probe.

According to another aspect of the present invention there is provided an apparatus for directing a treatment tool towards an organic target located within a body cavity, comprising an elongated member having a distal portion insertable into a body cavity, the elongated member comprises a channel sized to accommodate a treatment tool, the channel has a distal opening within the distal portion of the elongated member, and a pre-bent treatment tool having a distal portion sized to fit within the channel. The tool and the channel are so sized and so configured that the tool can be inserted into the channel while the distal portion of the elongated member is inserted in a body cavity, the tool can be advanced within the channel while the distal portion of the elongated member is so inserted, and the distal portion of the tool assumes a bent configuration when the distal portion of the tool is advanced beyond the distal opening of the channel. In a preferred embodiment the elongated member further comprises a lumen sized to accommodate a visual guiding apparatus such as an optical hysteroscope.

In a preferred embodiment, the elongated member comprises a plurality of channels each sized to accommodate a treatment tool. In a further preferred embodiment the apparatus comprises a plurality of pre-bent treatment tools and the tools comprise markings showing information describing the pre-bent tools. Preferably the markings are positioned on a proximal portion of each pre-bent tool and are operable to be visible to an operator when a distal portion of the pre-bent tool is inserted in the channel. In an additional preferred embodiment, the treatment tools comprises markings which show what length of distal portion of the tool extends beyond a distal opening of the channel when the tool is inserted through the channel and extends beyond a distal opening of the channel.

According to yet another aspect of the present invention there is provided an apparatus for steering a treatment probe to a treatment target within a body cavity, comprising a delivery guide having a channel sized to accommodate a treatment probe, the channel comprises a proximal portion, a distal portion, and a pivot joining the proximal portion to the distal portion, the pivot enabling variability in angular positioning of the distal portion with respect to the proximal portion.

According to further features in preferred embodiments of the invention described below, the apparatus further comprises a maneuvering member by which an operator may control the angular positioning while the distal portion of the delivery guide is inserted within a body of a patient. The apparatus preferably comprises a treatment probe sized to be insertable in the channel and having an operating tip operable to treat the treatment target. The probe preferably comprises a shaft having a flexible proximal portion and a rigid distal portion. The probe may be a cryoprobe. In a preferred embodiment the delivery guide further comprises a lumen sized to accommodate a visual guiding apparatus, preferably an optical hysteroscope. Optionally, the delivery section comprises a plurality of pivots.

According to a still further aspect of the present invention there is provided a method for delivering a plurality of treatment probe heads to a treatment target within a body cavity, comprising:

(a) providing a visual guiding apparatus, a first treatment probe having a first treatment head operable to treat the treatment target, a second treatment probe having a second treatment head operable to treat the treatment target, and a sleeve configured to accommodate the visual guiding apparatus and at least one of the first and second treatment probes, the sleeve having an opening running along its length and having a distal portion insertable into a body cavity;

(b) utilizing the visual guiding apparatus to guide placement of the first treatment head of the first treatment probe at a position appropriate for treating the treatment target;

(c) freeing the first treatment probe from the sleeve;

(d) inserting the second treatment probe into the sleeve; and

(e) utilizing the visual guiding apparatus to guide placement of the second treatment head at a position appropriate for treating the treatment target.

According to yet a further aspect of the present invention there is provided amethod for delivering a plurality of treatment probe heads to a treatment target within a body cavity, comprising:

(a) providing a visual guiding apparatus, a plurality of treatment probes each having a treatment head operable to treat the treatment target, and a sleeve sized to accommodate the visual guiding apparatus and at least one of the plurality of treatment probes, the sleeve having an opening running along it's length and having a distal portion insertable into a body cavity;

(b) inserting the visual guiding apparatus and a first treatment probe into the sleeve and inserting the distal portion of the sleeve into the body cavity;

(c) utilizing the visual guiding apparatus to guide placement of a treatment head of the first treatment probe at a position appropriate for treating the treatment target;

(d) removing the visual guiding apparatus from within the sleeve;

(e) displacing the sleeve so as to free the first treatment probe from containment within the sleeve;

(f) reinserting the visual guiding apparatus into the sleeve and into the body cavity;

(g) inserting a second treatment probe into the sleeve and into the body cavity; and

(h) utilizing the visual guiding apparatus to guide placement of the second treatment probe at a position appropriate for treating the treatment target.

Preferably, at least one of the first and second treatment probes is a cryoprobe.

According to further features in preferred embodiments of the invention described below, the method further comprises cooling the first treatment probe, and thereby causing the first treatment probe to adhere to the treatment target, before displacing the sleeve to free the first treatment probe from the sleeve. Alternatively, the treatment head of the first treatment probe may be attached to the target using mechanical means such as a hook, before removing the visual guiding apparatus from within the body cavity.

In a preferred embodiment the sleeve is at least partially constructed of heat-insulating material. In a preferred embodiment the method further comprises positioning within a cervix a portion of the sleeve, which portion comprises heat-insulating material, and maintaining the portion positioned within the cervix while using a treatment probe to cool a treatment target within a uterus. It is also recommended to utilize the sleeve to introduce a heat source into a body cavity, and using the heat source to heat first tissues within the body cavity while utilizing the cryoprobe to cool second tissues within the body cavity. The heat source may be, for example, a warm-water balloon.

An additional enhancement comprises inserting at least one cryogen supply tube attached to at least one treatment probe through a heat-insulating sleeve prior to insertion of the treatment probe into the treatment target, and positioning the heat-insulating sleeve within a cervix prior to thermal operation of the treatment probe. Additional methods for installing a heat-insulating sleeve over at least one cryogen supply tube attached to at least one treatment probe, and positioning the heat-insulating sleeve within a cervix prior to thermal operation of the treatment probe, include using an insulating sleeve with a slit, and using an insulating sleeve which is openable and closeable.

According to yet another aspect of the present invention there is provided a method for inserting multiple treatment probes into a target tissue within a body cavity, comprising the steps of:

(a) supplying a first treatment probe which comprises:
(i) a shaft having a maximum diameter D, the shaft being sized and shaped to enable passage of the treatment probe through a working channel of an endoscope;
(ii) a treatment head connected to a distal portion of the shaft and operable to treat a target tissue within a body cavity when supplied with a material substance transported to the treatment head through the shaft; and
(iii) a connector positioned at a proximal portion of the shaft and operable to connect the shaft to a source of the material substance, the connector having a diameter not substantially superior to the diameter D of the shaft;

(b) inserting the first treatment probe into working channel of an endoscope;

(c) inserting the endoscope into the body cavity and positioning the treatment head of the first treatment probe in a vicinity of the target tissue;

(d) disconnecting the connector from the source of material substance, if connected;

(e) retracting the endoscope from the body cavity while leaving the treatment head of the first probe positioned at the target tissue, the shaft of the first probe transiting the working channel of the endoscope and exiting from a distal end of the working channel as the endoscope is retracted;

(f) inserting a second treatment probe into the working channel of the endoscope and re-inserting the endoscope into the body cavity; and

(h) positioning a distal portion of the second treatment probe in a vicinity of the target tissue.

It is recommended to anchor the first treatment probe to the target tissue prior to retracting the endoscope from the body cavity. One may anchor the first treatment probe to the target tissue by cooling a portion of the probe adjacent to the target tissue, thereby causing the tissue to freeze and to adhere to the probe.

In a preferred embodiment, at least one of the first and second probes is a cryoprobe and the endoscope is a hysteroscope.

The endoscope may comprise a single working channel. Alternatively, the endoscope comprises a sleeve having a lumen for a visual guiding apparatus and a plurality of working channels sized to accommodate treatment probes.

The present invention successfully addresses the shortcomings of the presently known configurations by providing an apparatus and method enabling effective, rapid, convenient and thorough treatment of therapeutic ablation targets within body cavities, and in particular of uterine fibroids positioned within the uterus or embedded in the uterine wall.

The present invention further successfully addresses the shortcomings of the presently known configurations by providing an apparatus capable of passing easily through a dilated cervix, yet operable to deliver to a target fibroid a plurality of treatment probes in a configuration appropriate for treating a large fibroid whose external dimensions largely exceed the diameter of the dilated cervix.

The present invention further successfully addresses the shortcomings of the presently known configurations by providing an apparatus and method for sequentially delivering a plurality of treatment probes through a common channel to a common treatment target, whereat said plurality of probes may be operated simultaneously.

The present invention further successfully addresses the shortcomings of the presently known configurations by providing an apparatus and method providing enhanced maneuverability of thermal treatment probes used in the uterus.

The present invention further successfully addresses the shortcomings of the presently known configurations by providing an apparatus and method enabling cryoablation within the uterus while protecting tissues of the cervix from damage by cold.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified view of tools and techniques used to treat uterine fibroids, according to methods of prior art;

FIG. 2(a) is a line drawing of components of resectoscopic equipment currently in use in the treatment of fibroids, according to methods of prior art;

FIG. 2(b) is a line drawing of a simple sleeve resectoscope comprising a hysteroscope inserted into sleeve, according to methods of prior art;

FIG. 2(c) is a line drawing of a more complex sleeve resectoscope comprising manipulation actuators, according to methods of prior art;

FIG. 3(a) is a simplified schematic of a thermal ablation system, according to an embodiment of the present invention;

FIG. 3(b) is a simplified schematic presenting an expanded view of an endoscopic sleeve shown in FIG. 3(a), according to an embodiment of the present invention;

FIGS. 4(a)-4(d) are simplified schematics of a pre-bent treatment probe and of an apparatus for inserting that pre-bent probe into a treatment target such as a uterine fibroid, according to an embodiment of the present invention;

FIGS. 5(a)-5(c) are simplified schematics of a steering apparatus for steering a flexible treatment probe, according to an embodiment of the present invention;

FIG. 6(a) is a simplified schematic showing a cross-sectional view of an apparatus for delivering a plurality of cryoablation needles to a cryoablation target, according to an embodiment of the present invention;

FIG. 6(b) is a simplified schematic showing a side view of an apparatus for delivering a plurality of cryoablation probes to a cryoablation target, according to an embodiment of the present invention;

FIGS. 6(c)-6(e) are simplified schematics showing progressive stages of use of the apparatus presented by FIGS. 6(a) and 6(b), according to an embodiment of the present invention;

FIGS. 6(f)-6(h), are simplified schematic views of an apparatus for cryoablation by multiple cryoprobes within a uterus, comprising a feature which protects the cervix from damage by cold generated during the cryoablation process, according to an embodiment of the present invention;

FIGS. 6(i) and 6(j) are simplified schematic views of closed and open configurations respectively of an apparatus for delivering a plurality of treatment probes to a treatment target, according to an embodiment of the present invention;

FIGS. 7(a) and 7(b) are simplified schematics of a multi-probe delivery system, according to an embodiment of the present invention;

FIGS. 7(c) and 7(d) are simplified schematics of an asymmetric multi-probe delivery system, according to an embodiment of the present invention;

FIG. 8 is a simplified schematic showing a side-view cross-section of a sleeve for delivering a plurality of treatment probes to a treatment target, according to an embodiment of the present invention;

FIGS. 9(a) and 9(b) are simplified schematics of a treatment probe having a low-profile connector, in disconnected and connected configurations respectively, according to an embodiment of the present invention;

FIGS. 10(a) and 10(b) are simplified schematics of a probe having low profile connector designed for insertion into a scope having a working channel, according to an embodiment of the invention;

FIG. 11 is a simplified schematic showing the apparatus of FIG. 10 used to deliver a plurality of treatment probes to a common ablation target, according to an embodiment of the present invention;

FIG. 12(a) is a simplified schematic of a treatment probe insertion and manipulation apparatus, according to an embodiment of the present invention;

FIG. 12(b) is a simplified schematic showing a side view of the apparatus presented in FIG. 12(a), according to an embodiment of the present invention;

FIG. 13(a) is a simplified schematic of a heat insulator for use in cryosurgery, according to an embodiment of the current invention;

FIG. 13(b) is a simplified schematic showing a cross-sectional view of a slit heat insulator inserted in a cervix opening, according to an embodiment of the present invention; and

FIG. 13(c) is a simplified schematic showing a cross-sectional view of a heat insulator having a split configuration inserted into a cervix opening, according to an additional embodiment of the present invention.

### DESCRIPTION OF THE PREFERRED EMBODIMENTS

The present invention is of apparatus and methods for delivering thermal treatment probes to a treatment target within a body cavity, and in particular to delivery of a plurality of cryoprobes to a fibroid within a uterus, thereby enabling thermal ablation of the fibroid. Specifically, the present invention enables to deliver a plurality of thermal treatment probes through the cervix into the uterus, and there to deploy those probes in a dispersed configuration appropriate for thermal treatment of a large fibroid.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrase “heat-exchanging configuration” is used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, CO₂, CF₄, and xenon, and various other gasses pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which have the property of becoming hotter when passed through a Joule-Thomson heat exchanger. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating.

The terms “ablation temperature” and “cryoablation temperature”, as used herein, relate to the temperature at which cell functionality and structure are destroyed by cooling. According to current practice temperatures below approximately −40° C. are generally considered to be ablation temperatures.

The term “ablation volume”, as used herein, is the volume of tissue which has been cooled to ablation temperatures by one or more cryoprobes.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

The terms “thermal ablation system” and “thermal ablation apparatus”, as used herein, refer to any apparatus or system useable to ablate body tissues either by cooling those tissues or by heating those tissues.

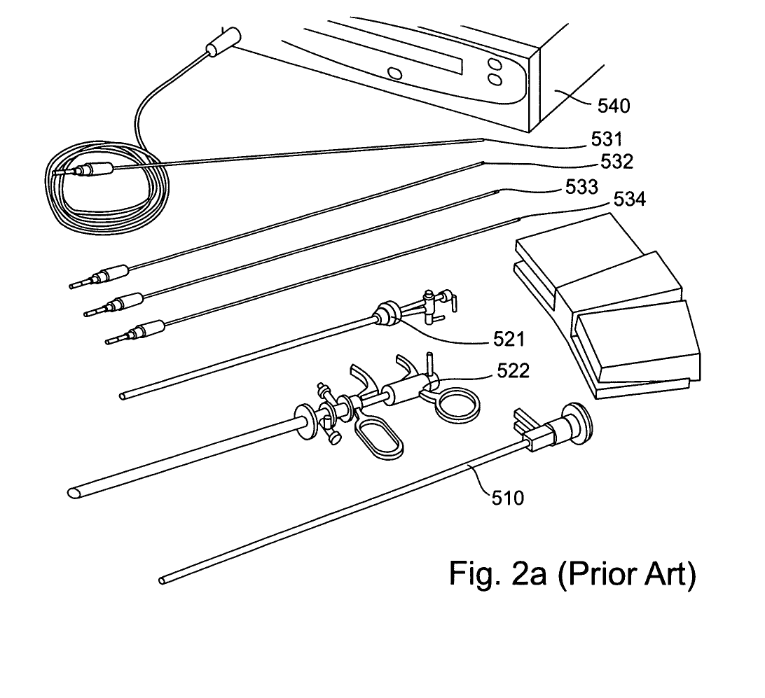

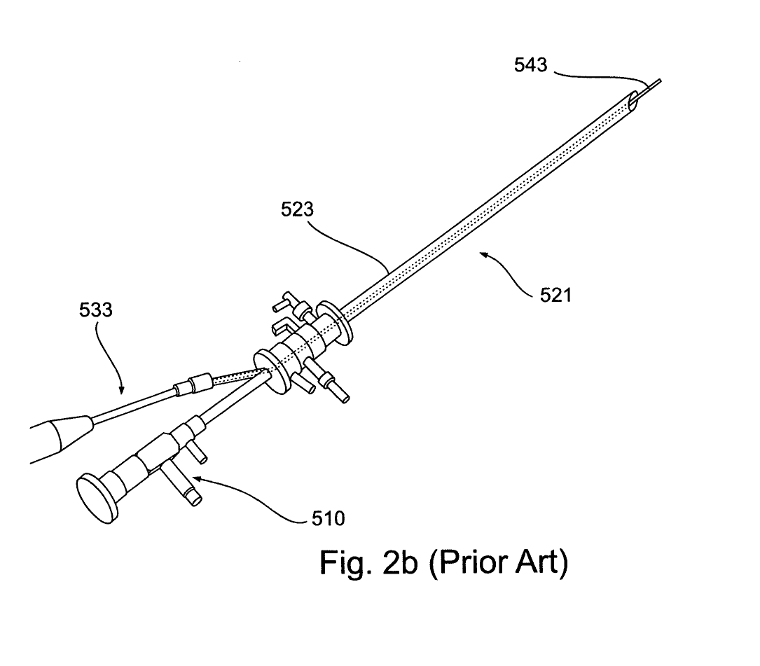

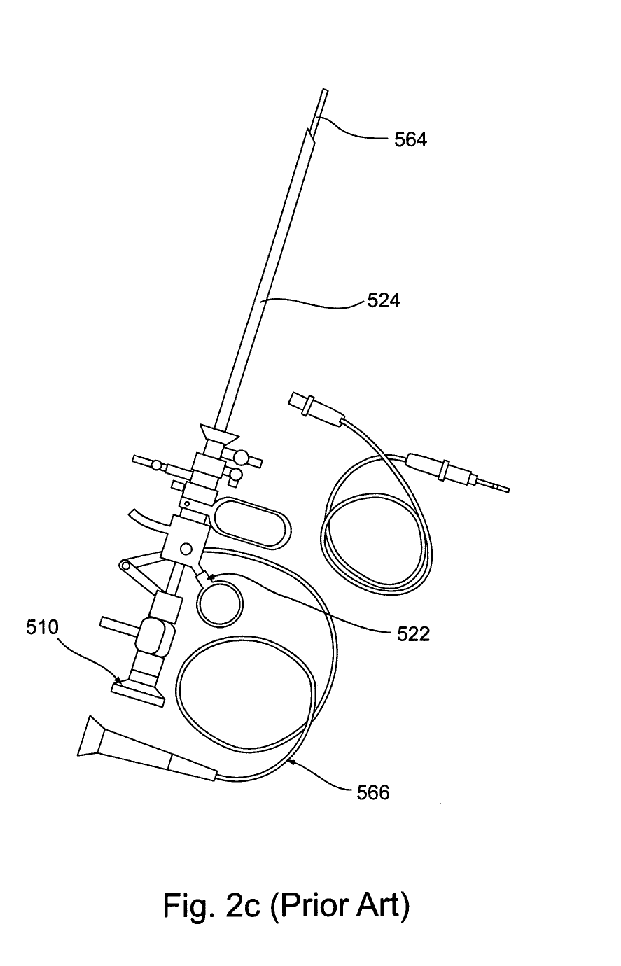

The term “optical hysteroscope” is used herein to refer to optical equipment comprising a flexible portion operable to be inserted into a uterus and providing means for visual inspection of interior surfaces of that uterus by a surgeon or other operator. The term “treatment hysteroscope” is used herein to refer to surgical equipment such as that represented in FIGS. 2(a)-2(c) and discussed hereinbelow, wherein a device comprises both an optical hysteroscope as defined above and also a working channel thorough which a treatment probe or other surgical equipment may be introduced into a uterus or other body cavity. Typically, when a distal portion of a treatment hysteroscope is inserted in a body cavity such as a uterus, and a surgical device such as a treatment probe is inserted through the working channel of that treatment hysteroscope, a distal portion of the inserted surgical device may be observed in relationship to portions of the interior of that body cavity by means of the optical hysteroscope portion of the treatment hysteroscope.

For exemplary purposes, the present invention is principally described in the following with reference to an exemplary context, namely that of treatment of fibroids within a uterus. It is to be understood that invention is not limited to that exemplary context. The invention is, in general, relevant to treatment of any surgical target within any natural or man-made body cavity, including the uterus but not limited thereto. Thus, methods and devices of the present invention are relevant to treatment of the rectal cavity, of airways, of the esophagus, and a variety of other similar bodily contexts. Similarly, although the following discussion is primarily addressed to the exemplary context of hysteroscopic equipment and operations, the present invention is to be understood to be directed as well to the more general context of endoscopic equipment and operations of any sort. Thus, references to “hysteroscope” herein should be understood to apply as well to endoscopes in general.

It is expected that during the life of this patent many relevant cryoprobes and cryoprobe conduits will be developed, and the scope of the terms “cryoprobe” and “cryoprobe conduit” is intended to include all such new technologies a priori.

Similarly, it is expected that during the life of this patent many relevant hysteroscopes and endoscopes will be developed, and the scope of the terms “hysteroscope” and “endoscope” is intended to include all such new technologies a priori.

As used herein the term “about” refers to ±10%.

In discussion of the various figures described hereinbelow, like numbers refer to like parts.

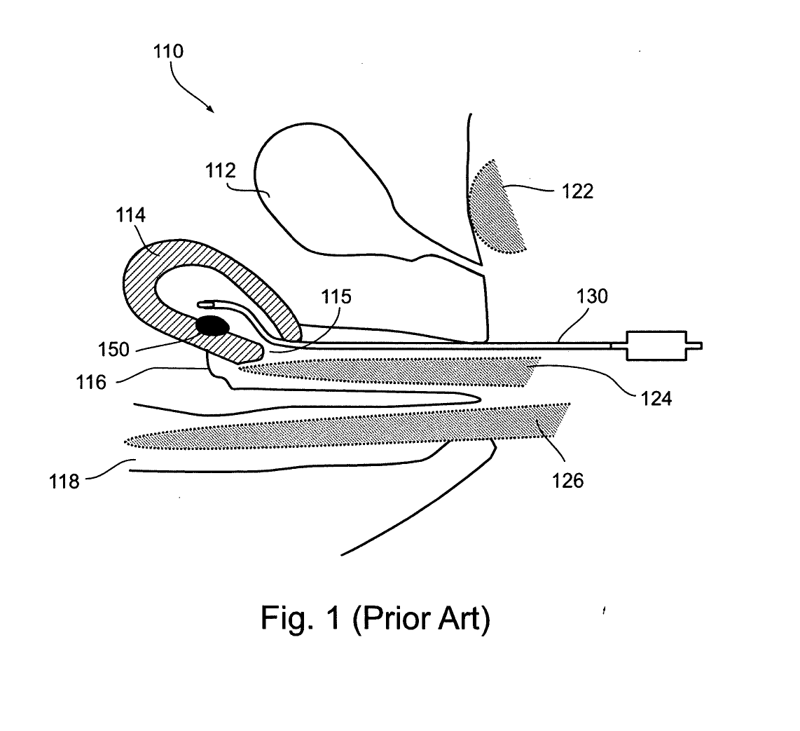

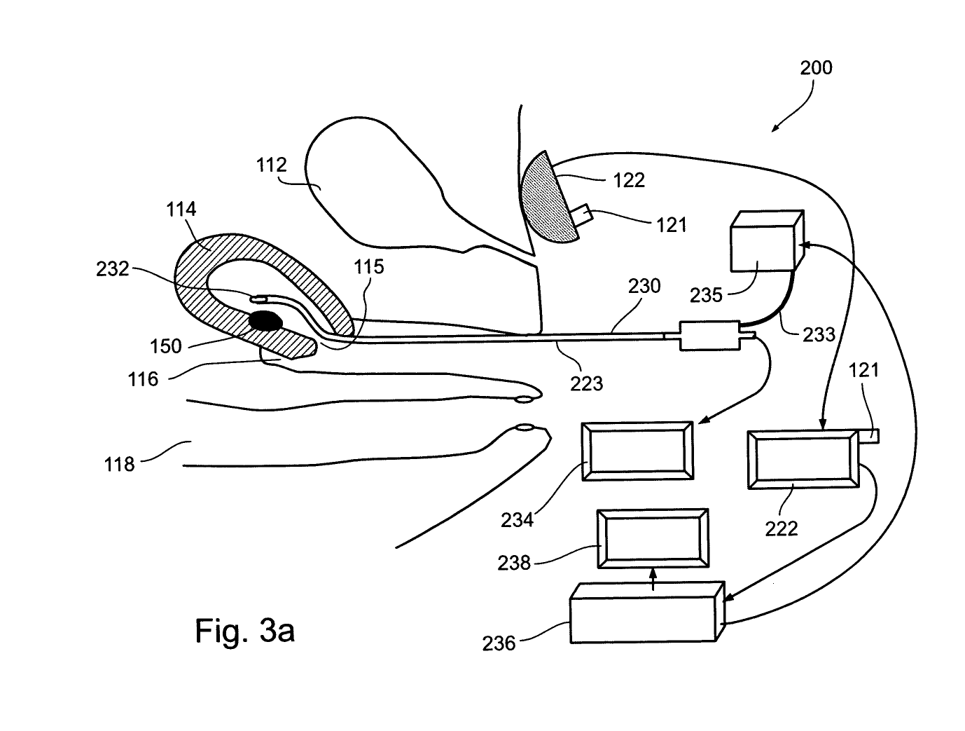

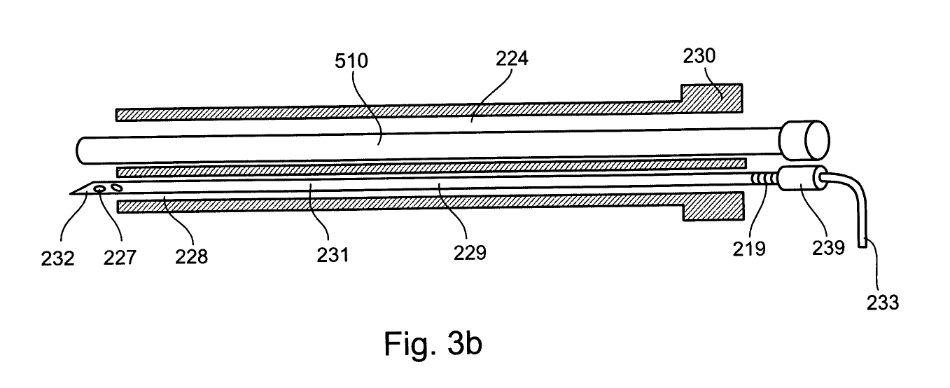

For purposes of better understanding the present invention, as illustrated in FIGS. 3-13(c) of the drawings, reference is first made to conventional (i.e., prior art) tools and techniques used in the treatment of uterine fibroids, as illustrated in FIGS. 1, 2(a), 2(b) and 2(c).

Attention is now drawn to FIG. 1, which presents a simplified view of tools and techniques used to treat uterine fibroids, according to methods of prior art. FIG. 1 presents a mid-sagittal cross section of a women patient 100. A bladder 112, uterus 114, vagina 116, and rectum 118 may be seen. A fibroid 150 may be seen within uterus 114.

Several viewing and imaging devices may be used to observe and diagnose the fibroid. An ultrasound apparatus may be used with one of the several types of probe. An abdominal ultrasound probe 122 may be positioned outside the body and in contact with the abdomen. A vaginal ultrasound probe 124 may be inserted into vagina 116. A rectal ultrasound probe 126 may be inserted into rectum 118. Alternatively or additionally, a hysteroscope 130 may be inserted into the uterine cavity through the cervix 115 for visual viewing of the fibroid 150.

Attention is now drawn to FIG. 2(a), which is a line drawing derived from a photograph of resectoscopic components currently in use in the treatment of fibroids, according to methods of prior art.

FIG. 2(a) presents an optical hysteroscope 510 which may be used to view the inner lumen of the uterus. In use, optical hysteroscope 510 is typically inserted into the internal bore of one of sleeve 521 or of sleeve 522. One of electrodes 531, 532, 533 or 534, shown in the Figure, may be connected via a cable to electrode control box 540, and passed through a working channel in sleeve 521 or sleeve 522 when the sleeve is inserted through the cervix. The inserted electrodes can then be used for ablation of uterine tissue. The combination of sleeve 521 (or sleeve 522) with optical hysteroscope 510 thus constitutes a treatment hysteroscope as defined hereinabove.

Attention is now drawn to FIG. 2(b), which is a line drawing of a simple treatment hysteroscope 523, also referred to as a sleeve resectoscope, comprising optical hysteroscope 510 inserted into sleeve 521. Electrode 533 is also shown inserted into sleeve 521 through a working channel of sleeve 521. Operating tip 543 of electrode 533 is seen protruding from the distal end of sleeve 521, where it could be used to ablate uterine tissue.

Attention is now drawn to FIG. 2(c), which is a line drawing based on a photograph of a more complex sleeve resectoscope (treatment hysteroscope) 524 which comprises manipulation actuators. In FIG. 2(c) optical hysteroscope 510 is seen inserted into sleeve 522. Sleeve 522 comprises manipulation actuators, whose operating handles may be seen in the Figure. An electrode is shown inserted into sleeve 522. Power cable 566 is attached to a proximal end of the inserted electrode, and operating tip 564 of the inserted electrode may be seen protruding from the distal tip of sleeve 522, in which position operating tip 564 might be used to ablate uterine tissue.

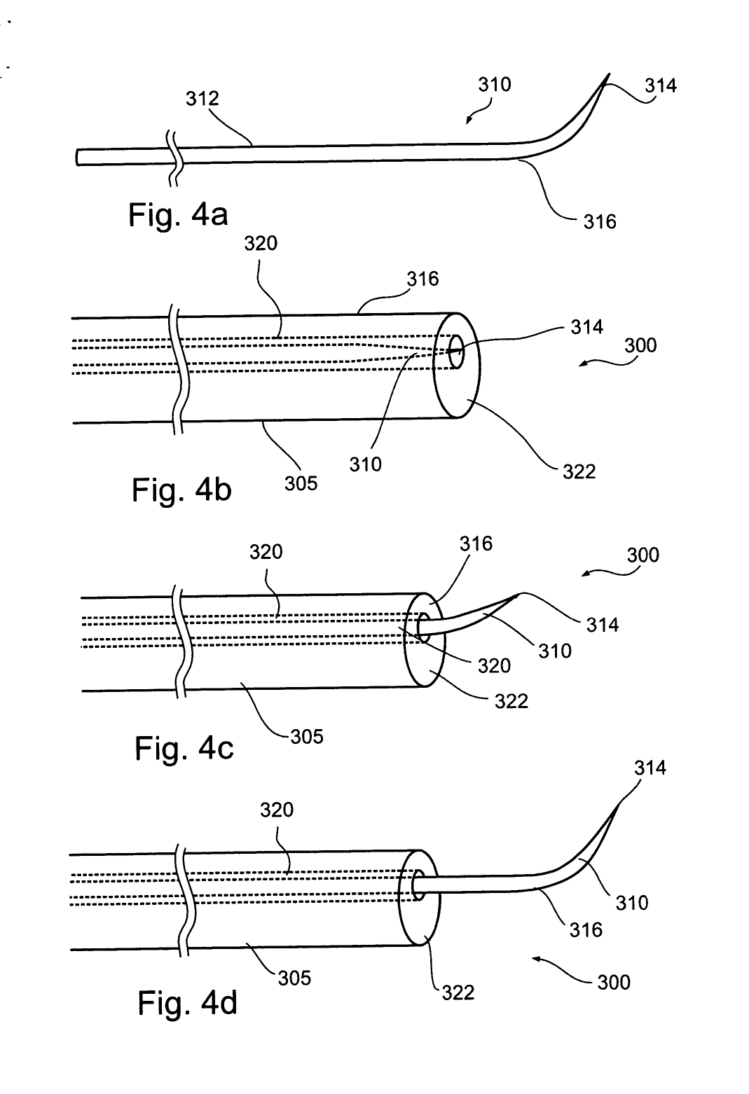

Attention is now drawn to FIG. 3(a), which is a simplified schematic of a thermal ablation system, according to an embodiment of the present invention. FIG. 3(a) presents an exemplary context within which various embodiments presented hereinbelow with reference to FIGS. 4-13(c) may optionally be embodied, yet it is to be understood that the context presented in FIG. 3(a) is provided by way of example and is not intended to be limiting: embodiments of the invention presented by FIGS. 4-13(c) and discussed hereinbelow may be implemented in a variety of other contexts and of other systems for treating surgical targets within body cavities.

FIG. 3(a) presents a thermal ablation system 200 for treating tissues (for example, uterine fibroids) within a confined body cavity. Fibroid treatment system 200 comprises an endoscopic sleeve 223, here presented (by way of example) as a hysteroscopic sleeve 230 inserted into a uterus 114 through a cervix 115.

Attention is also drawn to FIG. 3(b), which is a simplified schematic presenting a more detailed view of sleeve 230. As may be seen in FIG. 3(b), sleeve 230 comprises a lumen 224 sized to accommodate an optical hysteroscope 510, and also comprises at least one working channel 228 through which a treatment probe 231 having an operating tip 232 may be inserted. In FIG. 3(a) a single working channel 228 is shown, yet various embodiments presented hereinbelow comprise multiple working channels 228 and/or single working channels 228 sized and configured to accommodate multiple treatment probes 231.

In a preferred embodiment probe 231 is a cryoprobe 229 or other type of thermal ablation probe, and a cryogen connector 239 is provided on a proximal portion of probe 231 for connecting probe 231 to a cryogen supply hose 233. Probe 231 may be, for example, a Joule-Thomson cryoprobe cooled by Joule-Thomson cooling of expanding high-pressure gas. In that case, cryogen supply hose 233 will supply high-pressure cooling gas (such as argon) to probe 231. Alternatively, probe 231 may be a cryoprobe cooled by evaporation of a liquid cryogen. In that case, cryogen supply hose 233 will supply liquid cryogen such as liquid nitrogen to probe 231.

Probe 231 is preferably flexible or semi-rigid, and sleeve 230 and optical hysteroscope 510 are similarly preferably flexible or semi-rigid.

In FIG. 3(a), a flexible thermal treatment probe 231 is shown inserted through working channel 228 of sleeve 230. (Since in FIG. 3(a) sleeve 230 is presented as opaque, only operating tip 232 of inserted probe 231, and hose 233 used for cryogen delivery to inserted probe 231, are visible in the Figure). When sleeve 230, optical hysteroscope 510 and treatment probe 231 are inserted as shown in FIG. 3, optical hysteroscope 510 enables visual inspection of the interior of uterus 114, and further enables guidance of thermal treatment probe 231 towards a uterine ablation target such as fibroid 150. Optical hysteroscope 510 may also be used to guide placement of an optional thermal sensing probe (not shown in this Figure), useful for controlling thermal ablation processes.

Thermal treatment probe 231 (typically a thermal ablation probe) is used for delivering or absorbing thermal energy at its tip 232. Thermal ablation probe 231 is preferably a cryoprobe 229 capable of freezing and destroying fibroid cells and optionally also capable of being heated in order to quickly thaw or otherwise heat tissue. In a preferred embodiment cryoprobe 229 is a Joule-Thomson cryoprobe operable to cool to cryoablation temperatures by Joule-Thomson cooling, that is, by expansion of high-pressure cooling gas, and preferably also operable to heat tissues by Joule-Thomson heating, that is, by expansion of high pressure heating gas.

Optionally, sleeve 230 may be a rigid hysteroscope such as those known to prior art. Thus, hysteroscope/sleeve combinations shown in FIGS. 2(b) and 2(c) might be used.

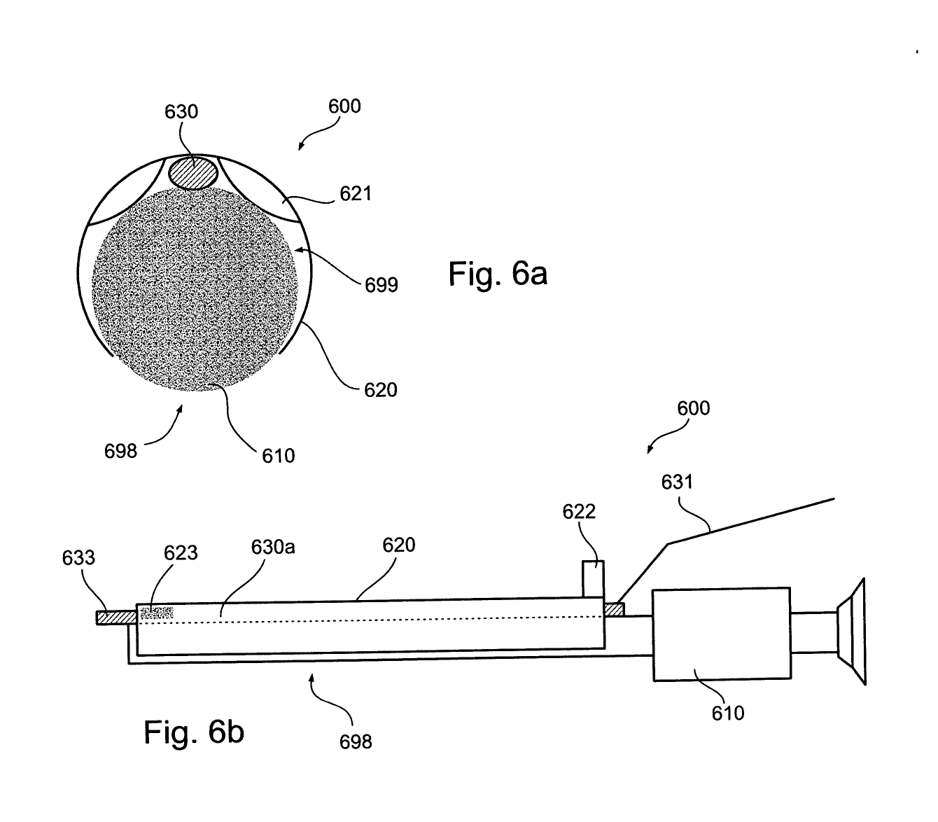

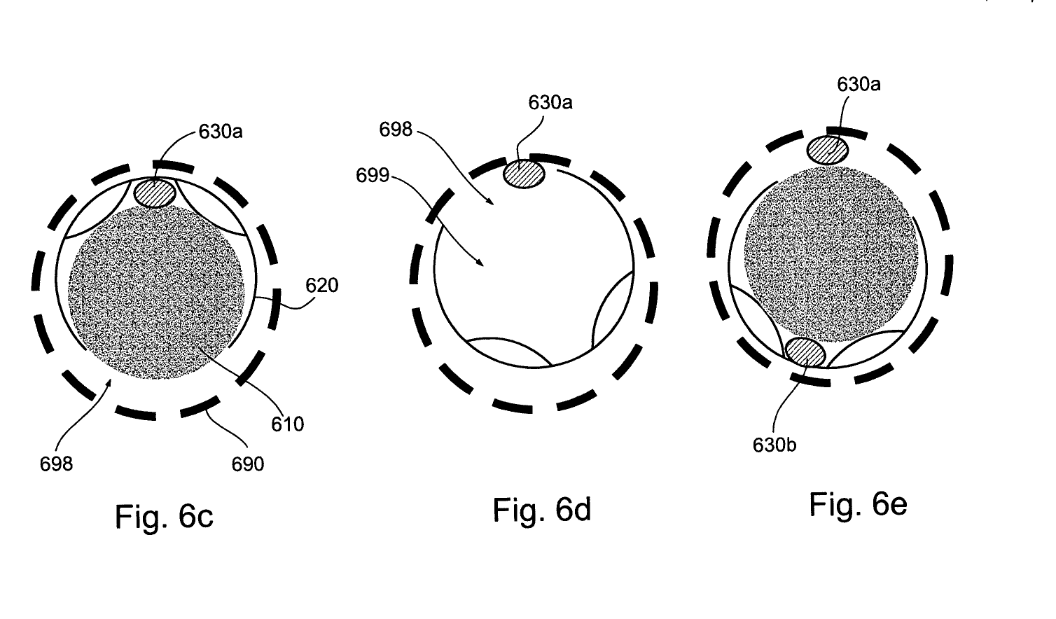

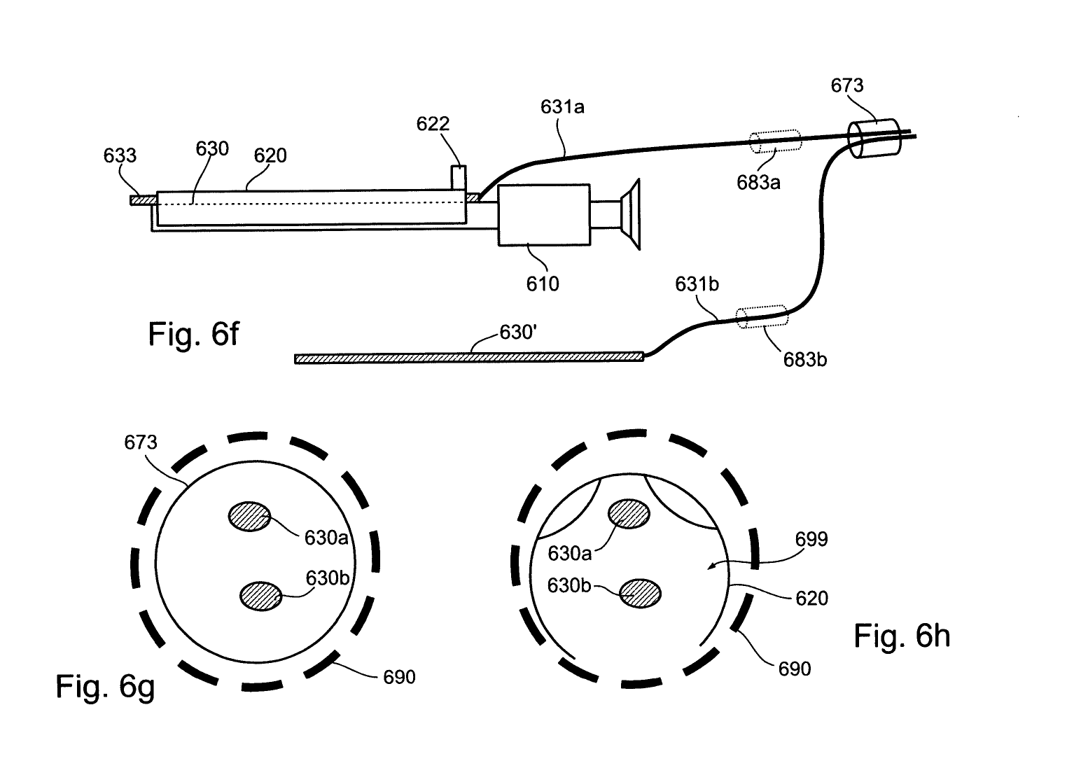

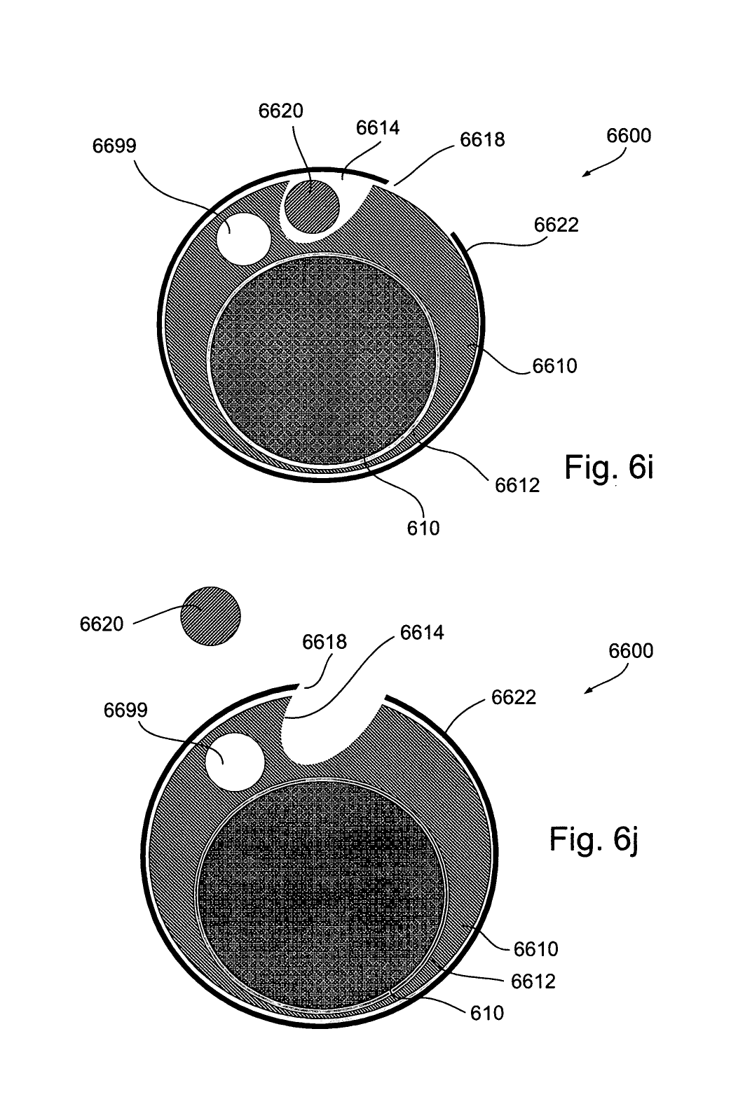

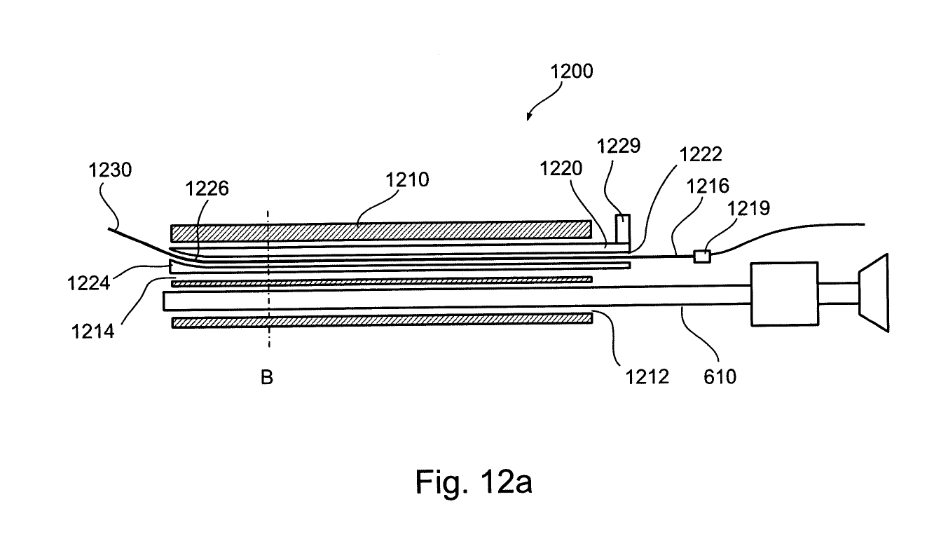

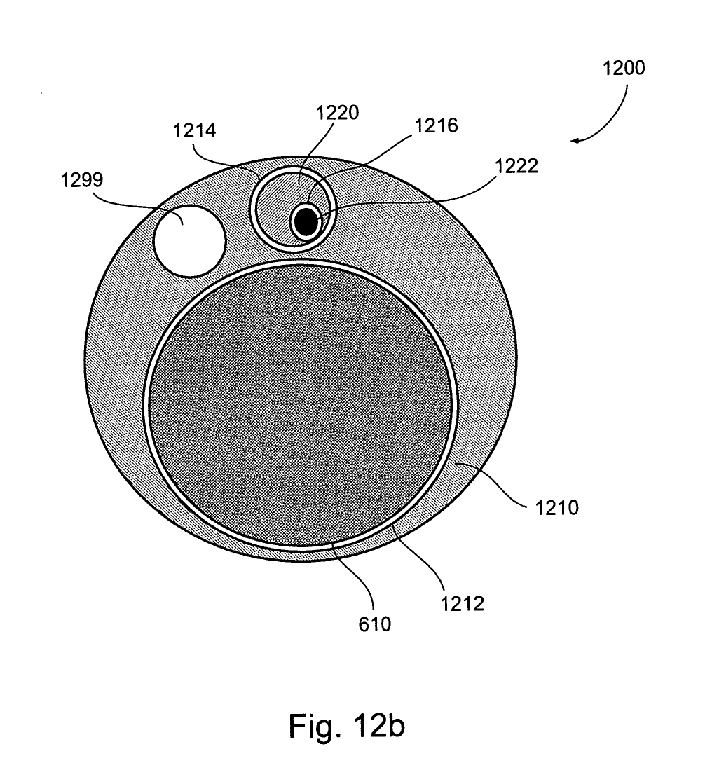

Preferably, however, a sleeve adapted for guidance of multiple probes should be used. Details of such hysteroscope units are disclosed hereinbelow and discussed with particular reference to FIGS. 6(a), 6(b), 6(c) 7(a), 7(b), 8, 10, 11 and FIG. 12.

As shown in FIG. 3(a), an optional hysteroscope control and display unit 234 may be used to view images of the uterine interior. This feature is well known and commonly available in fiber-optic and television camera hysteroscopes. Using control and display unit 234, the user is enabled to view the interior of the uterus and to observe a uterine treatment target such as fibroid 150 before, during, and after insertion and operation of cryoprobe 229, or before, during, and after operation of any other thermal treatment probe 231

In an additional preferred embodiment an abdominal ultrasound probe 122 is used to image fibroid 150. (Note that fibroid 150 is referenced herein as an exemplary ablation target. Reference to fibroid 150 should be understood as relating also to any appropriate target of ablative surgery.) Abdominal ultrasound probe 122 is connected to an ultrasonic control unit 222 which powers probe 122 and processes its signals. Ultrasonic control unit 222 may be any commercially available ultrasound unit equipped with a display (not shown in the Figure).

Ultrasound imaging module 122/222 may be used to image fibroid 150 prior to surgery, in order to assess size and position of fibroid 150. In cryosurgery, such an assessment is used during a treatment planning stage, prior to actual cryosurgery, to determine the size of ice ball required to ensure effective treatment of the target. Calculating size of a required iceball enables selection of appropriate types and numbers of cryoprobes to be used, and enables planning position and depth to which each cryoprobe is inserted.

In a preferred embodiment of the present invention cryoprobe(s) 229 or other thermal treatment probes 231 comprise an echogenic section 227 which serves to enhance visibility of probe 231 by ultrasound imaging module. 122/222. Ultrasound imaging module 122/222 is preferably used to monitor position and size of ice balls formed during cryoablation treatment, and helps to determine when to halt treatment to protect vital organs such as the cervix, rectum, bowels, etc. Abdominal ultrasound probe 122 may be moved to various positions in order to change viewpoints or to observe areas obscured by rigid and non-ultrasonically-transparent items such as hysteroscope 230, cryoprobe 229, or ice balls created by operation of cryoprobes 229.

In a preferred embodiment, abdominal ultrasound probe 122 is equipped with a location sensor 121, such as the electromagnetic location sensor “CARTO™ XP EP Navigation and Ablation System” sold by Biosense Webster (Israel) Ltd, Tirat Carnel; ISRAEL, which may be seen at www.biosensewebster.com. Alternatively, any of the electromagnetic or electromechanical locations sensors well known in the art may be used. Location sensor 121 provides information on the position and direction from which various image views are taken, thereby providing information regarding the spatial relationships between objects visible in disparate views. Such information enables to register ultrasonic images taken by moveable ultrasound probe 122 within a common fixed Cartesian coordinate system. Use of such a common coordinate system enables, for example, comparing of actual visible ice-ball location and size to planned ice-ball location and size. A common coordinate system is particularly important in cases where ultrasound probe 122 is moved during monitoring, and in cases where images taken for planning purposes are taken from a different viewing point from that used during surgery, or made by a different imaging device.

As shown in FIG. 3(b), in a preferred embodiment, distance markings 219 are provided on a proximal portion of the shaft of treatment probe 231 (which may be a cryoprobe 229), which markings serve to display the depth to which a distal end of probe 231 extends beyond hysteroscope 230, which distance is correlated with a distance by which probe 231 is inserted into a treatment target such as fibroid 150. Thus, depth of insertion of probe 231 into a target such as a fibroid may be inferred from observation of markings on a proximal portion of shaft of probe 231 extending from a proximal end of sleeve 230.

As shown in FIG. 3(a), in a preferred embodiment, ultrasound images produced by ultrasound imaging module 122/222 are relayed to a thermal ablation control unit 236, which unit is used for planning and monitoring thermal treatment. Preferably, thermal ablation control unit 236 is operable to overlay a simulated (planned) treatment result on an actual real-time ultrasound image obtained during surgery, and further operable to display a resultant combined image on a display 238, thereby enabling clear visual comparison between planned and actual thermal ablation.

When probe 231 is a cryoprobe 229, a cryogen control unit 235 is provided to control supply of cryogen to cryoprobe 229, and thereby to control cooling and optionally heating of cryoprobe 229. If cryoprobe 229 is a Joule-Thomson cryoprobe, cryogen control unit 235 will be a controller operable to control supply of high-pressure cooling gas and optionally high-pressure heating gas. Cryogen control unit 235 supplies cryogen through hose 233 to cryoprobe 229, where it traverses the shaft of cryoprobe 229 and is delivered to an operating tip 232 of cryoprobe 229, which tip is cooled by expansion of the cryogen (in the case of a Joule-Thomson cryoprobe) or by evaporation of the cryogen (in the case of an evaporative cryoprobe). Cryogen control unit 235 may be controlled by thermal ablation control unit 236, or alternatively may be manually controlled a user.

It is noted that embodiments presented by FIGS. 4-13(c) and discussed in detail hereinbelow may be implemented in various forms and used in a variety of contexts. However, in preferred embodiments, features and devices presented hereinbelow are embodied within, and used in conjunction with, thermal ablation system 200 as presented in FIG. 3 and described hereinabove.

Attention is now drawn to FIGS. 4(a)-4(d), which present simplified schematics of a pre-bent treatment probe and an apparatus for inserting that pre-bent probe into a treatment target such as a uterine fibroid, according to an embodiment of the present invention.

Uterine fibroids located in a hard-to-reach locations, for example uterine fibroids located near the cervix, are an example of a class of hard-to-reach treatment targets which may be successfully reached and treated utilizing pre-bent treatment probes, as presented by FIG. 4.

FIG. 4 presents a probe delivery apparatus 300 operable to deliver a pre-bent probe 310 to a treatment target. Pre-bent probe 310 may be probe 231 of FIG. 3(a), or any thermal ablation probe or other treatment probe. In a preferred embodiment, pre-bent probe 310 is a cryoprobe 229, and most preferably a Joule-Thomson cryoprobe as described hereinabove. Alternatively, probe 310 may be an evaporative cryoprobe. Probe delivery apparatus 300 comprises pre-bent probe 310 and an elongated member 305 containing a delivery channel 320 having a distal opening 322.

Elongated member 305 of probe delivery apparatus 300 may, for example, be a hysteroscope 230. Most hysteroscopes are equipped with straight working channel, which limits their ability to deliver a thermal ablation needle into a fibroid located near the cervix or in various other inconvenient locations. To overcome this limitation, pre-bent probe 310 may be used as shown in FIG. 4, with working channel 228 of hysteroscope 230 serving as probe delivery channel 320 as shown in FIG. 4. Thus, delivery channel 320, embodied as working channel 228 of hysteroscope 230, is operable to deliver pre-bent cryoprobe 310 through distal opening 322 of delivery channel 320. In this embodiment, distal opening 322 is preferably positioned within the viewing range of hysteroscope 230.

A pre-bent thermal ablation probe 310 having a semi-rigid shaft 312, a thermal tip 314 and a bend 316 may be seen in FIG. 4(a). Fabrication processes known in the art enable to fabricate thermal ablation probe 310 (e.g. of stainless steel) with a desired bent shape and a desired degree if springiness, so as to be flexible yet tending to spring back to a pre-determined bent shape. For convenient surgical practice it will be preferable to make available to a surgeon a plurality of pre-bent probes 310, configured with a variety of bending angles and lengths of arc, from which plurality of probes a surgeon can select the probe or probes most appropriate for a particular task at hand. In a preferred embodiment, information about direction, radius, and length of bend is printed on the shaft of each probe 310, preferably on a proximal portion of the shaft so as to be visible to an operator when probe 310 is inserted in channel 320.

If probe 310 is a cryoprobe, shaft 312 is connectable by flexible hose to a cryogen control unit 235 operable to regulate supply of a cryogen to probe 310.

Pre-bent probe 310 is semi-rigid and can be straightened and inserted into delivery channel 320 as seen in FIG. 4(b). Shaft 312 is sufficiently strong that pushing on its proximal end causes its distal end to extend from delivery channel 320, thereby enabling insertion of sharpened thermal tip 314 into tissue, for example into fibroid 150.

Optionally, pre-bent probe 310 may be rotated inside delivery channel 320, so that bend 316 may be directed towards a desired direction.

As shown in FIGS. 4(c) and 4(d), a selected length of distal portion of probe 310 may be extended beyond the distal end of delivery channel 320, resulting in a selected degree of curvature of the exposed distal portion of probe 310. A distal portion of probe 310 of selected length may be extended from delivery channel 320 before sharpened thermal tip 314 is introduced into a target tissue such as fibroid 150, or alternatively delivery channel 320 may be positioned directly on a target tissue such as fibroid 150, and then an arc of probe 310 of selected length may be extended within that target tissue. It is anticipated that, for target tissues with the toughness of a fibroid, positioning delivery channel 320 contiguous to the fibroid prior to advancing probe 310 from channel 320 will generally be found to be a preferable method for introducing sharpened thermal tip 314 into such a target. Markings on the proximal shaft of probe 310 may be provided, which markings serve to indicate what distal length of probe 310 extends beyond distal opening 322 of delivery channel 320 at any given time.

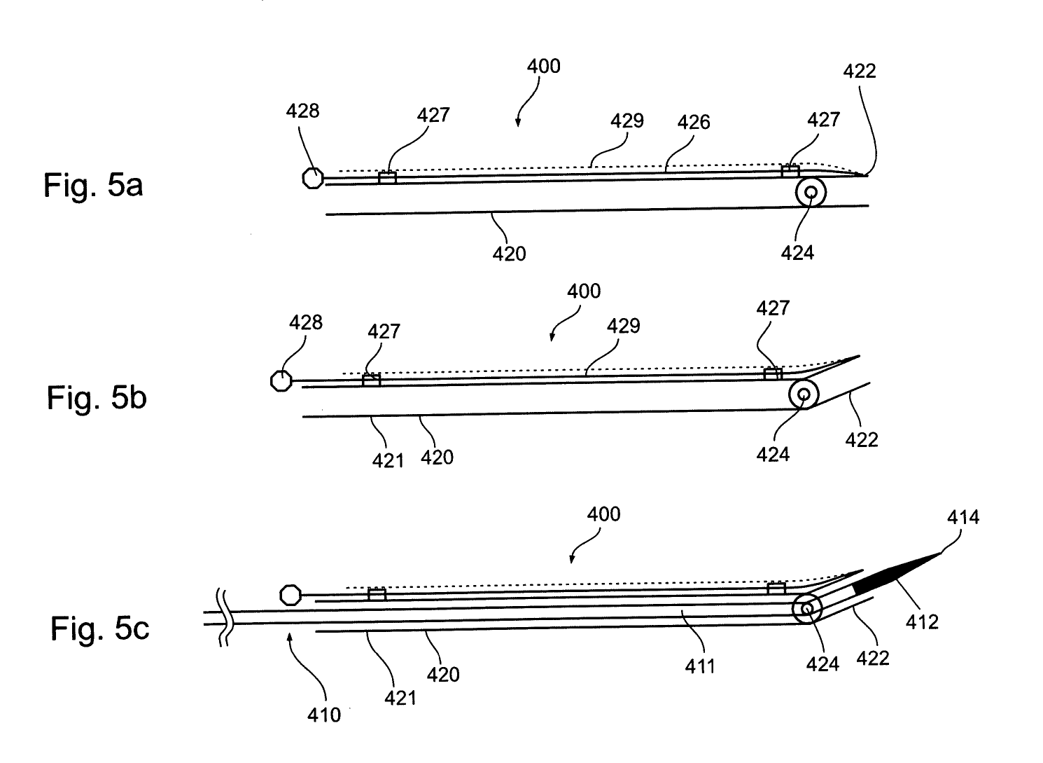

Attention is now drawn to FIGS. 5(a)-5(c), which present a steerable flexible-probe delivery apparatus 400 which comprises a treatment probe 410 and a steerable delivery guide 420 operable to steer flexible treatment probe 410 to a treatment target, according to an embodiment of the present invention.

Steerable delivery guide 420 may be implemented as a stand-alone elongated member operable to be inserted into a body cavity. Alternatively, delivery guide 420 may be implemented as a working channel within an elongated member having additional features and functions. In particular, in a preferred embodiment steerable flexible-probe delivery apparatus 400 is embodied as a feature of a hysteroscopic sleeve 230, which sleeve also comprises a lumen for an optical hysteroscope 510, use of which enables steering flexible treatment probe 410 towards and into a treatment target such as uterine fibroid 150 under hysteroscopic-enabled visual observation.

As shown in FIG. 5(a), steerable delivery guide 420 comprises a proximal stationary section 421 and a distal movable section 422 joined by a pivot 424. (Of course, stationary section 421 is not stationary in an absolute sense, since apparatus 400 is itself moveable. However, when apparatus 400 is held immobile at its proximal end (for example, when apparatus 400 is inserted into a body cavity of a patient), stationary section 421 will be held immobile, yet moveable section 422 may be maneuvered to a variety of positions.)

A maneuvering member 426, for example a cable, connects movable section 422 to maneuvering handle 428 so that by exerting force on maneuvering handle 428, the relative angle between stationary section 421 and movable section 422 may be controlled. Holders 427 hold maneuvering member 426 to stationary section 421 in such manner that maneuvering member 426 is able to slide freely within holders 427. Preferably, a cover 429, capable of flexing when movable section 422 is actuated, covers both holders 427 and maneuvering member 426, to prevent injury to the cervix during insertion of apparatus 400 through a cervix into a uterine cavity.

FIGS. 5(a) and 5(b) depict steerable delivery guide 420 in straight and bent configurations respectively.

Maneuvering member 426 may be embodied as a cable or plurality of cables, optionally pulling against a spring (not shown) acting on movable section 422.

Optionally, steerable delivery guide 420 may be rotated so that movable section 422 may be oriented in a desired direction.

Optionally, guide 420 may comprise a plurality of movable sections and pivots, so as to be able to form an arch when manipulated.

Thermal treatment probe 410 may be a cryoprobe 229 (Joule-Thomson cryoprobe or evaporative cryoprobe) as described hereinabove. Thermal treatment probe 410 preferably comprises a shaft with a flexible section 411 and a rigid section 412 leading to a sharp operating tip 414. In a recommended method of operation, an operator inserts probe 410 into steerable delivery guide 420, and shaft 411 is pushed forward into guide 420 until a desired length of rigid section 412 is exposed as depicted in FIG. 5(c). The operator then controls the direction of tip 414 by operating maneuvering handle 428 and optionally by rotating delivery guide 420. When tip 414 is appropriately positioned and oriented, an operator then inserts tip 414 into target tissue (such as fibroid 150) by further pushing proximal shaft 411 of probe 410 into delivery channel 420.

In a preferred embodiment, probe 410 is implemented as a cryoprobe 229, and steerable flexible-probe delivery apparatus 400 is embodied in a hysteroscopic sleeve 230. Shaft 411 of probe 410 is connectable by flexible hose to a cryogen control unit 235 operable to regulate supply of a cryogen to probe 410. In a preferred embodiment, cryogen control unit 235 is controlled by thermal ablation control unit 236, as shown in FIG. 3(a) and described hereinabove.

In a recommended method of operation when apparatus 400 is embodied as a hysteroscopic sleeve 230, an operator inserts probe 410 into target tissue as described above, and then retracts sleeve 230 and/or optical hysteroscope 510, leaving probe 410 in position to ablate fibroid 150 or another ablation target, while protecting the sensitive optics of optical hysteroscope 510 from adverse effects of temperature extremes produced during thermal ablation. If probe 410 is implemented as a cryoprobe, it is recommended that an operator activate probe 410 for a short duration and/or under low cooling settings prior to retracting hysteroscope 510 and/or sleeve 230, in order to freeze tissues immediately adjacent to probe 410, thereby causing probe 410 to adhere to treated tissues, before retracting sleeve 230 and/or hysteroscope 510. Retraction of sleeve 230 after positioning probe 410 is preferably facilitated by use of a low-profile connector to connect probe 410 to a cryogen source. Low-profile connecters are discussed in detail hereinbelow.

In alternative modes of use, one or several apparatus 400 may be used to deliver one or more flexible straight probes to a treatment target, to deliver one or more pre-bent probes to a treatment target, or to deliver to a treatment target a desired combination of straight and pre-bent probes.

In general, it is preferable to bring steerable delivery channel 420 to a straight configuration before retracting probe 410.

Attention is now drawn to FIGS. 6(a) and 6(b), which are a simplified schematics of front and side views of an apparatus for delivering a plurality of cryoablation needles to a cryoablation target, and to FIGS. 6(c)-6(e) which present progressive steps in the utilization of the apparatus of FIGS. 6(a) and 6(b), according to an embodiment of the present invention.

Hysteroscopes and resectoscopes currently in use allow only a single thermal ablation probe to be brought to bear on a fibroid or other uterine treatment target. However, some fibroids are larger than the maximum size of ice balls which can be created using small-diameter cryoprobes. Moreover, in typical use, each cryoablation cycle requires up to thirty minutes to complete. Thus, it is advantageous to be able to bring to bear on a large target (such as a large uterine fibroid) a plurality of probes operable to perform cryoablation (or other thermal ablation) concurrently.

FIGS. 6(a)-6(e) present such a system. System 600 is operable to deliver a plurality of thermal treatment probes to a treatment target within a body cavity (such as a uterine fibroid within a uterus), which probes may then be used to perform concurrent cryoablation. FIG. 6(a) shows a cross-sectional view of system 600, and FIG. 6(b) presents a side view of system 600.

System 600 comprises a sleeve 620, a visual guiding apparatus 610 and a plurality of treatment probes 630, labeled 630a and 630b in the Figures. Visual guiding apparatus 610 may be an optical hysteroscope or any other visual guiding apparatus. Probes 630 are any embodiments of treatment probe 231 as described hereinabove, and are preferably cryoprobes 229, cooled by Joule-Thomson cooling or by evaporative cooling.

Probe 630 and visual guiding apparatus 610 are designed to fit within and slide freely through open channel lumen 699 of sleeve 620, when positioned within sleeve 620 as shown in FIG. 6(a). Sleeve 620 is shaped and dimensioned so as to be capable of insertion into a uterine lumen through a cervix 690, as seen in FIG. 6(c). For treating a fibroid, visual guiding apparatus 610 is preferably a hysteroscope. Sleeve 620 comprises common lumen 699 within which both visual guiding apparatus 610 and probe 630 can be positioned.

A distal portion of sleeve 620 can be inserted into a uterus through cervix 690. Before or after such insertion either or both of visual guiding apparatus 610 and probe 630 can be introduced into sleeve 620 and advanced therethrough. Thus, sleeve 620 serves as a conduit operable to deliver both visual guiding apparatus 610 and probe 630 to a treatment site, such as a fibroid, within a uterus.

Sleeve 620 is not wholly closed, but rather comprises an opening 698 running along its length, as seen in FIGS. 6(a) and 6(b).

Preferably, probe 630 is free to be pushed along sleeve 620 while visual guiding apparatus 610 is inserted in sleeve 620. Alternatively, probe 630 may be fixed to sleeve 620 or to visual guiding apparatus 610 when visual guiding apparatus 610 is installed within sleeve 620.

FIG. 6(c) shows probe 630 and visual guiding apparatus 610 installed in sleeve 620. In this condition, probe 630 may be brought into contact with a cryoablation target, and inserted therein. Visual guiding apparatus 610 may be pulled out of sleeve 620 at any time. In particular, visual guiding apparatus 610 may be pulled out of sleeve 620 while a distal portion of sleeve 620 is inserted within a uterine cavity and while needle 630 is inserted into a fibroid or other treatment target.

Once visual guiding apparatus 610 is removed from sleeve 620, probe 630 is free to move within common lumen 699, as depicted in FIG. 6(d). With probe 630 able to move freely within lumen 699, sleeve 620 may be freed from probe 630, for example by pulling sleeve 620 out of the uterine cavity, or by rotating sleeve 620 so that opening 689 is positioned facing probe 630, enabling probe 630 to exit sleeve 620 through opening 689, as shown in FIG. 6(d). Optional sleeve handle 622, connected to sleeve 620, may be used to facilitate manipulation of sleeve 620.

In a recommended mode of operation, probe tip 633 of a first probe 630a is pushed to protrude beyond the distal end of sleeve 620 and inserted into fibroid 150 or other treatment target. Visual guiding apparatus (e.g. hysteroscope) 610 is then pulled out of sleeve 620, as shown in FIG. 6(d), freeing probe 530. If probe 630a is a cryoprobe, it is recommended that probe 630a be operated at low power or for a short duration so as to freeze tissues immediately adjacent to probe tip 633, thereby anchoring probe 630a at its location (e.g. inserted into fibroid 150), before freeing probe 630a from sleeve 620.

Sleeve 620 may be removed from the uterus, refitted with visual guiding apparatus 610 and a second treatment probe 630b, and reintroduced into the uterine cavity as shown in FIG. 6(e), thereby enabling insertion of second treatment probe 630b into a second location in fibroid 150 (or into a second fibroid or other target) under optical guidance of visual guiding apparatus 610. Alternatively, sleeve 620 may be left inserted in the uterus as visual guiding apparatus 610 and second treatment probe 630b are inserted therein.

These steps may be repeated for insertion of additional treatment probes 630c, 630d, etc. as needed. A variety of therapeutic probes and tools may thus be introduced. For example, sleeve 620 might be used to introduce a probe 630 embodied as an ultrasonic probe into a uterus, for purposes of monitoring size and position of ice-balls created by therapeutic probes 630 embodied as cryoprobes. Sleeve 620 might similarly be used to introduce a warm-water balloon through a cervix for protecting cervix or portions of a uterus during cryoablation of a fibroid or other uterine target. Sleeve 620 may also be used without presence of visual guiding apparatus 610, to introduce and position any other instrument which (because of size or for any other reason) cannot be introduced while visual guiding apparatus 610 present in sleeve 620. Sleeve 620 may also be used and then removed, to introduce into a body cavity any instrument which may conveniently be introduced into that cavity by use of sleeve 620, but which cannot well be used while sleeve 620 is present (as might be the case, for example, with placement and use of an intra-uterine ultrasound probe).

Sleeve 620 is so dimensioned that a plurality of probes 630 together with sleeve 620 may be accommodated within a dialated cervix opening 690. A typical optical hysteroscope has a diameter of 4 to 6 mm, while a thermal treatment probe 630 may have a diameter of 1 to 2 mm. In comparison, cervix opening 690 may be dilated to a diameter of 7 mm to 9 mm.

Optionally, treatment probes 630 may be constructed having a short rigid distal section near tip 633, and a long proximal shaft embodied as a flexible hose 631. By planning an appropriate order of needle insertion into fibroid 150 and by appropriately rotating and manipulating sleeve 620, a skilled surgeon using apparatus and methods presented in FIGS. 6(a)-6(e) and described hereinabove will be enabled to insert a plurality of treatment probes 630 into selected portions of treatment targets within a uterus, thereby enabling full and simultaneous treatment of all portions of a large fibroid or of multiple fibroids.

Sleeve 620 may be constructed of metal or of plastic, and may be designed for re-sterilization (appropriate for multiple uses) or as a disposable sleeve (preferably sterilely packaged) for one-time use.

Optionally, sleeve 620 may comprise channels 621 (shown in FIG. 6(a)) for irrigation of the field of view, for inflating the uterus, for circulating hot fluid to protect non-treated sections during cryoablation, and for various other uses. A plurality of additional working channels may be provided to accommodate for various additional tools. Sleeve 620 preferably also comprises echogenic surfaces 623 for enhanced ultrasound visibility.

Attention is now drawn to FIGS. 6(f)-6(h), which are simplified schematic views of an apparatus for cryoablation within a uterus by multiple cryoprobes, comprising a feature which protects the cervix from damage by cold generated during the cryoablation process, according to an embodiment of the present invention.

A heat-insulating sleeve 673 may be used as a component of system 600, to prevent damage to the cervix during cryoablation of a fibroid. In a recommended mode of operation, gas supply hoses 631 (labeled 631a and 631b in FIG. 6(f)) supplying high-pressure cooling gas to, and exhausting cold low-pressure cooling gas from, treatment probes 630 embodied as cryoprobes 229, are threaded through a common heat-insolating sleeve 673 before insertion into sleeve 620, as depicted in FIG. 6(f). Heat-insolating sleeve 673 remains on flexible gas hoses 631 until all treatment probes 630 are inserted into (and preferably cooled so as to adhere to) their fibroid targets. At that time heat-insolating sleeve 673 is pushed into position at cervix 690, as show in FIG. 6(g), where it remains during cryoablation and protects cervix 690 from damage by cold.

Alternatively, each of gas hoses 631 (631a, 631b, etc.) may be threaded through an individual heat-insulating sleeve 683 (labeled 683a and 683b in FIG. 6(f)). Individual heat-insulating sleeves 683 are then pushed along hoses 631 to protect cervix 690 (preferably after insertion of probes 630 into fibroid 150 and their adhesion thereto) and caused to remain in cervix 690 during the thermal ablation process.

Alternatively, open channel sleeve 620 may used for cervical protection as depicted in FIG. 6(h). According to this embodiment, after an operator has inserted all thermal treatment probes, he removes visual guiding apparatus 610 (typically an optical hysteroscope) from sleeve 620. The operator then repositions sleeve 620 in cervix 690 such manner that shafts of all inserted thermal treatment probes 630 are inside common lumen 699 of sleeve 620 and are not in contact with cervix 690, thereby protecting cervix 690 during cryoablation or other thermal treatment of fibroids or other uterine treatment targets. Preferably, at least a distal portion of sleeve 620 may be constructed of insulating material to reduce transfer of heat between cervix 690 and contents of sleeve 620.

Attention is now drawn to FIGS. 6(i) and 6(j), which are simplified schematics of an apparatus for delivering a plurality of treatment probes to a treatment target, the apparatus comprising a treatment probe lumen switchable between open and closed configurations, according to an embodiment of the present invention.

FIG. 6(i) presents an apparatus 6600 having a body 6610 comprising a lumen 6612 for a visual guiding apparatus 610 or other tool, an optional utility lumen 6699 for irrigation, insertion of additional surgical tools, or other uses, and a treatment tool lumen 6614 sized to accommodate a treatment tool 6620 such as, for example, a Joule-Thomson cryoprobe. Apparatus 6600 further comprises an apparatus cover 6622 having a slit 6618. Cover 6622 is at least partially rotatable around body 6610, and operable to take on an open state, with slit 6618 aligned with treatement tool lumen 6614 and treatment tool 6614 enabled to enter or to leave lumen 6614, and a closed state with slit 6618 rotated away from treatment tool lumen 6614, thereby closing treatment tool lumen 6614 and preventing treatment tool 6620 from entering or leaving lumen 6614. FIG. 6(j) shows apparatus 6600 in open state, with slit 6618 and treatment tool lumen 6614 in aligned configuration, enabling free passage of treatment tool 6620 in and out of apparatus 6600. FIG. 6(i) shows apparatus 6600 in closed state, with slit 6618 and treatment tool lumen 6614 in unaligned configuration, preventing treatment tool from leaving or entering lumen 6614.

In a recommended method of use, apparatus 6600, supplied with a first treatment tool 6620, is visually guided (using visual guiding apparatus 610) to a first location where first treatment tool 6620 is inserted into target tissue. First tool 6620 is then preferably anchored at that first location, for example by a short application of cryocooling, thereby freezing tool 6620 to the target tissue.

Apparatus cover 6622 is then rotated until it is aligned with treatment tool lumen 6614, thereby freeing treatment tool 6620. As shown in the Figure, treatment tool lumen 6614 is preferably constructed in such a form that when cover 6622 is rotated to its open position, tool 6622 is easily released from apparatus 6600 by rotating apparatus 6600. As may be seen from the exemplary Figure, rotating apparatus 6600 counter-clockwise while a distal tip of treatment tool 6620 is anchored to target tissue, with a surgeon optionally holding and immobilizing a proximal part of treatment tool 6620, will cause tool 6620 to disengage from lumen 6614.

An operating surgeon then rotates cover 6622 to the position shown in FIG. 6(j) and inserts a second treatment tool 6620 into the now closed lumen 6614. That second tool may then be positioned with respect to a target tissue under guidance of visual guiding apparatus 610. These steps may then be repeated as desired, enabling an operator to inserting a plurality of treatment tools into a target tissue, one after another. Optionally, once this plurality of treatment tools is in place, apparatus 6600 may be removed from the body cavity before commencing ablation.

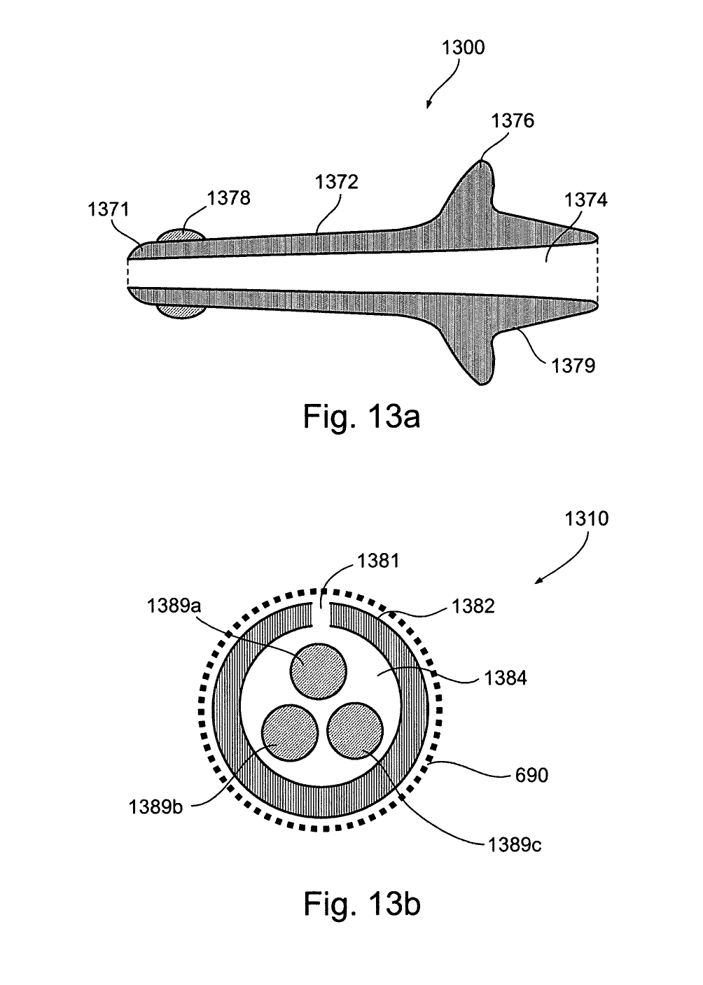

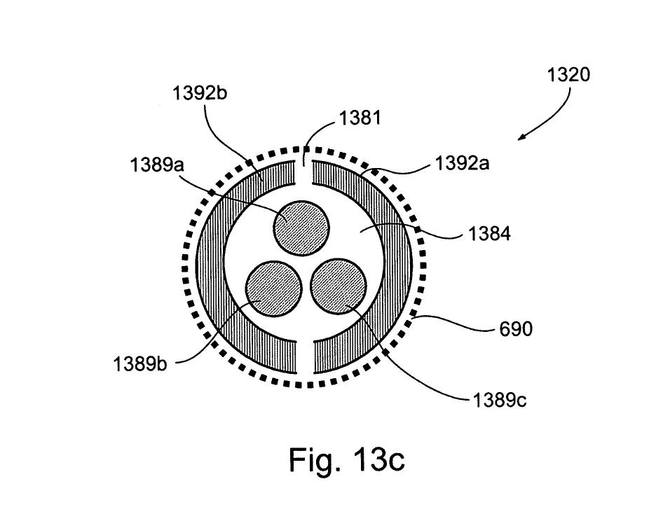

Optionally, heat insulating sleeves, discussed herein with respect to FIGS. 6f, 13a, 13b and 13c, may be utilized in conjunction with apparatus 6600 to protect tissues during ablation.

Optionally, visual guiding apparatus 610 may be integrated within body 6610 of apparatus 6600.

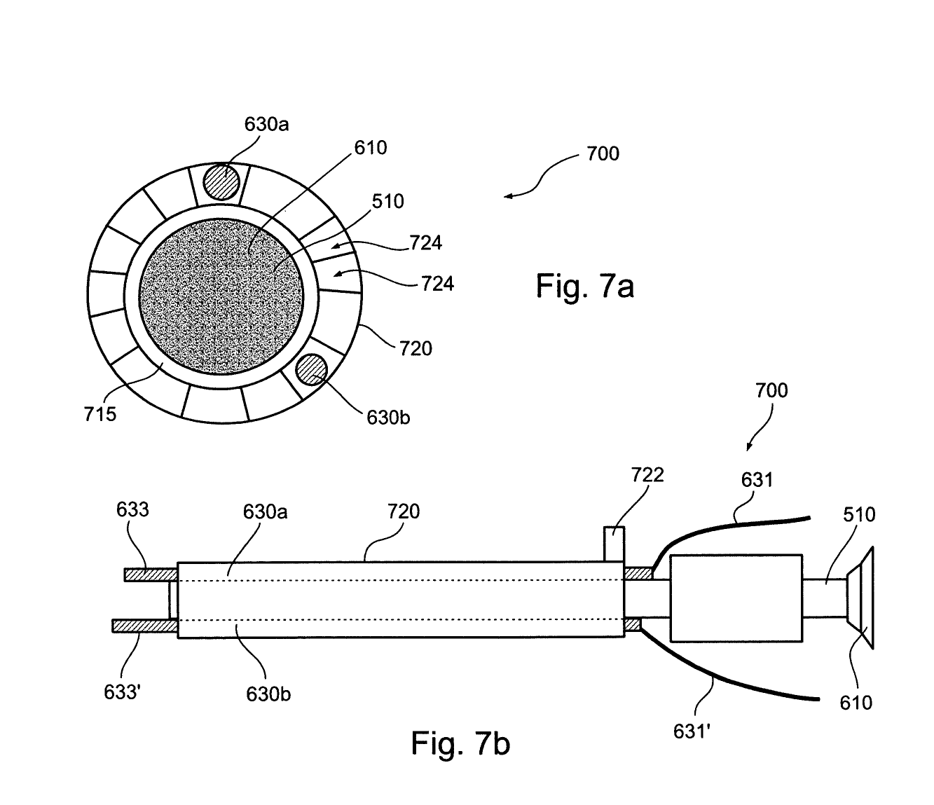

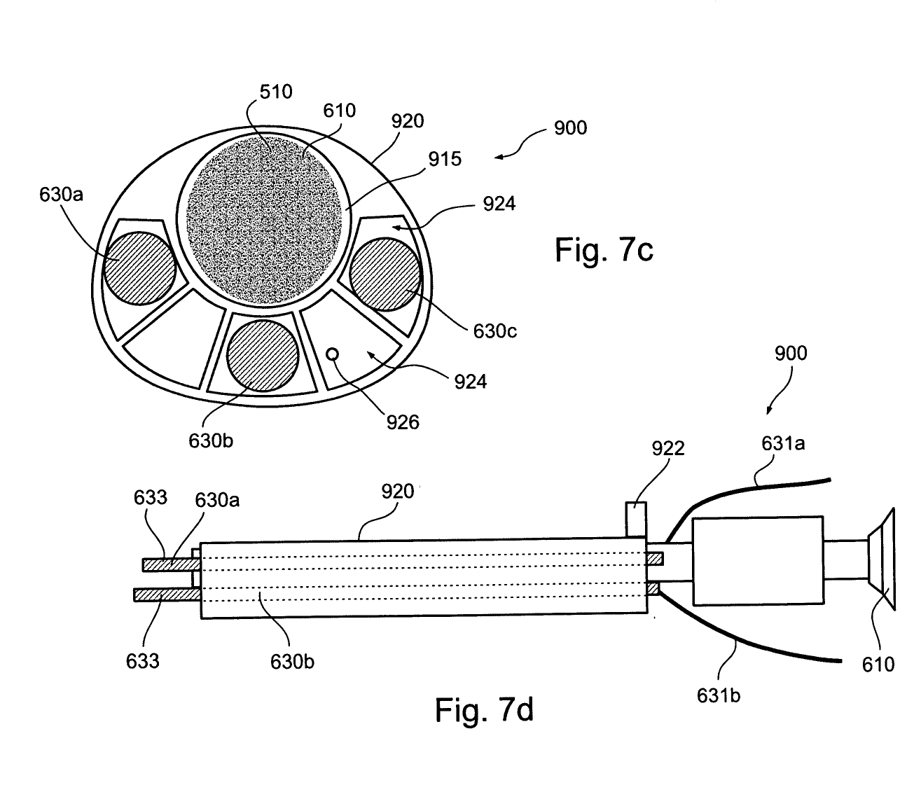

Attention is now drawn to FIGS. 7(a) and 7(b), which are simplified schematics of a multi-probe delivery system 700, according to an embodiment of the present invention. FIG. 7(a) provides a cross-sectional view of system 700. FIG. 7(b) provides a lateral view thereof.

System 700 comprises a tubular sleeve 720, a plurality of treatment probes 630, and a visual guiding apparatus 610 such as an optical hysteroscope 510. Sleeve 720 comprises a central lumen 715 sized to accommodate visual guiding apparatus 610, which is insertable into central lumen 715 of sleeve 720.

Sleeve 720 is sized so that its radius is larger (preferably only slightly larger) than the radius of visual guiding apparatus 610 plus a diameter of a treatment probe 630.

Sleeve 720 further comprises a plurality of channels 724 disposed around central lumen 715. At least some of channels 724 are sized to accommodate treatment probes 630, which are insertable into channels 724 and may be advanced therethrough until they protrude from a distal end of sleeve 720.

In a recommended method of use, each of a plurality of treatment probes 630 (examples are labeled 630a and 630b in the Figure) is advanced through a selected channel 724 of sleeve 720 after a distal portion of sleeve 720 is inserted into a body cavity (e.g., distal portion of sleeve 720 is inserted through a cervix into a uterus), until operating tips 633 of probes 630 extend beyond a distal end of sleeve 720 and into the body cavity, where they may be used to treat a treatment target. In a preferred embodiment of the present invention, treatment probes 630 are cryoprobes operable to cryoablate a fibroid. In a preferred mode of operation, a surgeon, having introduced sleeve 720 through a cervix into a uterus, and able to view the interior lumen of that uterus by means of visual guiding apparatus 610 inserted through central lumen 715 of sleeve 720, selects appropriate channels 724 to be used for insertion of a plurality of treatment probes 630, according to size and position of a treatment target, as seen by means of visual guiding apparatus 610, in relation the position of a distal portion of sleeve 720 in relation to that target. The surgeon may then view and guide insertion of operating tips 633 of treatment probes 630 into a selected treatment target such as a fibroid, and under some circumstances may continue to observe that target while effecting a thermal ablation procedure.

Preferably, treatment probes 630 are Joule-Thomson cryoprobes, as described hereinabove.

Optionally, sleeve 720 may be fitted with a handle 722, as shown in FIG. 7(b).

Optionally, some of channels 724 may be sized and otherwise optimized for additional purposes, such as for insertion of sensors or other tools for use at or near a treatment site, for irrigation of a treatment site to preserve clarity of field of view, for inflating a body cavity such as a uterus, for circulating hot fluid to protect non-treated sections of that cavity during thermal ablation, and for various other purposes.

Preferably, sleeve 720 comprises a heat-insulating material. As is well known in the art, cold expanded cooling gasses, after expanding in an expansion chamber of an operating tip of a Joule-Thomson cryoprobe and cooling that operating tip, continue to cool their neighborhood as they transit proximal portions (e.g. a shaft) of a treatment probe while exhausting therefrom. Cold expanded exhaust gasses can therefore cool proximal (shaft) portions of a cryoprobe and may damage tissues adjacent thereto. Cryoprobes cooled by evaporation of a cryogen may similarly damage tissues adjacent to proximal (shaft) portions of such probes, due to the extreme cold of evaporated cryogens exhausting from the treatment head of such probes. To prevent such tissue damage, heat-insulating material in sleeve 720 serves to thermally isolate proximal portions treatment probes 630, thereby preventing damage to the cervix and to other internal organs during cryoablation of fibroids or other treatment targets.

Sleeve 720 may be made of metal or plastic, and may be made to be sterilizable for multiple re-use, or alternatively may be produced in sterile disposable format appropriate for one-time use.

Pre-bent treatment probes, discussed hereinabove particularly in reference to FIG. 4, may be used with advantage when deployed through channels 724 of sleeve 720. In particular, a plurality of pre-bent treatment probes may be deployed through channels 724 in such orientation that treatment heads (thermal tips) 314 expand away from each other as they extend beyond sleeve 720, thereby providing a panoply of treatment heads having a diameter greater than the diameter of sleeve 720, which panoply of treatment heads may be appropriately sized and shaped for treating a large fibroid or other large treatment target.

Attention is now drawn to FIGS. 7(c) and 7(d), which are simplified schematics of an asymmetric closed multi-needle delivery system 900, according to an embodiment of the present invention. System 900 is presented in cross-sectional view by FIG. 7(c), and in lateral view in FIG. 7(d).

System 900 comprises an asymmetric closed sleeve 920 and visual guiding apparatus 610 such as a hysteroscope 510. Sleeve 920 comprises a first lumen 915 sized to accommodate visual guiding apparatus 610, and visual guiding apparatus 610 is insertable into first lumen 915 of sleeve 920, as shown in FIG. 7(c).

Sleeve 920 further comprises a plurality of channels 924 disposed asymmetrically in proximity to first lumen 915 of sleeve 920. At least some of channels 924 are sized to accommodate treatment probes 630, which are insertable into channels 924 and may be advanced therethrough until they protrude from a distal end of sleeve 920.

In a recommended method of use, each of a plurality of treatment probes 630 (examples are labeled 630a and 630b and 630c in FIG. 7(c) is advanced through a selected channel 924 of sleeve 920 while a distal portion of sleeve 920 is inserted into a body cavity such as a uterus, until operating tips 633 of probes 630 extend beyond a distal end of sleeve 920 and into the cavity, where they may be used to treat a treatment target. In a preferred embodiment of the present invention, treatment probes 630 are cryoprobes operable to cryoablate a fibroid. In a preferred mode of operation, a surgeon, having introduced sleeve 920 through a cervix into a uterus, and able to view the interior lumen of that uterus by means of visual guiding apparatus 610 inserted through central lumen 915 of sleeve 920, selects appropriate channels 924 to be used for insertion of a plurality of treatment probes 630, according to size and position of a treatment target, as seen by means of visual guiding apparatus 610, in relation the position of a distal portion of sleeve 920 in relation to that target. The surgeon may then view and guide insertion of operating tips 633 of treatment probes 630 into a selected treatment target such as a fibroid, and under some circumstances may continue to observe that target while effecting a thermal ablation procedure.

Preferably, treatment probes 630 are Joule-Thomson cryoprobes, as described hereinabove.

Optionally, sleeve 920 may be fitted with a handle 922, as shown in FIG. 7(d).

Optionally, some of channels 924 may be sized and otherwise optimized for additional purposes, such as for insertion sensors or other tools for use at or near a treatment site, for irrigation of a treatment site to preserve clarity of field of view, for inflating a uterus, for circulating hot fluid to protect non-treated sections of a uterus during cryoablation, and for various other purposes.

Preferably, sleeve 920 comprises a heat-insulating material. As is well known in the art, cold expanded cooling gasses, after expanding in an expansion chamber of an operating tip of a Joule-Thomson cryoprobe and cooling that operating tip, continue to cool their neighborhood as they transit proximal portions (e.g. a shaft) of a treatment probe while exhausting therefrom. Cold expanded exhaust gasses can therefore cool proximal (shaft) portions of a cryoprobe and may damage tissues adjacent thereto. Cryoprobes cooled by evaporation of a cryogen may similarly damage tissues adjacent to proximal (shaft) portions of such probes, due to the extreme cold of evaporated cryogens exhausting from the treatment head of such probes. To prevent such tissue damage, heat-insulating material in sleeve 920 serves to thermally isolate proximal portions treatment probes 630, thereby preventing damage to the cervix and to other internal organs during cryoablation of fibroids or other treatment targets.

Sleeve 920 may be made of metal or plastic, and may be made to be sterilizable for multiple re-use, or alternatively may be produced in disposable format for one-time use.

Pre-bent treatment probes, discussed hereinabove particularly in reference to FIG. 4, may be used with advantage when deployed through channels 924 of sleeve 920. In particular, a plurality of pre-bent treatment probes may be deployed through channels 924 in such orientation that treatment heads (thermal tips) 314 expand away from each other as they extend beyond sleeve 720, thereby providing a panoply of treatment heads having a diameter greater than the diameter of sleeve 920, which panoply of treatment heads may be appropriately sized and shaped for treating a large fibroid or other large treatment target.

In an exemplary embodiment of system 900 depicted in FIG. 7(d), sleeve 920 comprises five channels 924, of which three are used for thermal probes 630. However, number of channels 924 and number of treatment probes deployed therein may vary. Optionally, one or more thermal sensors 926 may be inserted into the body cavity under treatment through one or several of channels 924.

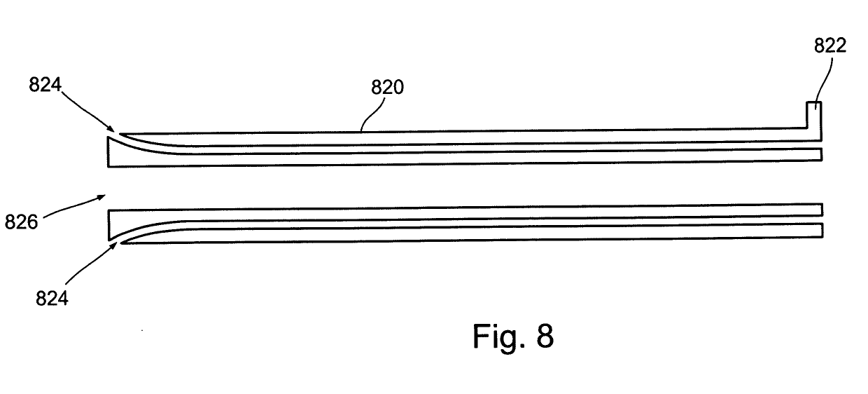

Attention is now drawn to FIG. 8, which is a simplified schematic of a side-view cross-section of a sleeve for delivering a plurality of treatment probes to a treatment target, according to an embodiment of the present invention.

The outer diameter of a sleeve appropriate for insertion into a uterus through a cervix is preferably less than 9 mm, due to limitations set by the maximum practical dilation of the cervical opening. Fibroids which it is desirable to treat, however, may have diameters of several centimeters.

Moreover, since size limitations of cervical openings limit the diameter of instruments designed to be inserted therethrough, it is highly preferable that thermal treatment probes designed to be inserted through a cervix (and in particular, those intended to be inserted through a cervix together with a hysteroscope), be of very small diameter, for example, 2 mm or 1.5 mm or less. Yet, cryoprobes of such limited cross-section have limited cooling capacities, because of gas flow restriction through gas supply and exhaust lumens of such small dimensions. Consequently, treating a large fibroid requires either a plurality of cryoprobes, or else a single probe used in a multi-stage treatment process which comprises freezing, thawing, repositioning of the probe or probes, re-cooling, etc., or both. Yet, treating in a multi-stage treatment process is inconvenient and time-consuming.

Sleeve 820 is operable to deliver a plurality of treatment probes, through a cervix, in a configuration which enables effective treatment of large fibroids in a single cooling cycle. Sleeve 820 may similarly be used in various other body cavities when it is desired to deliver a large spread of thermal treatment needles through a small opening.

FIG. 8 presents a side-view cross-section of a sleeve 820. Sleeve 820 may be sleeve 720 or sleeve 920 as described hereinabove, or any similar sleeve, but is characterized but having working channels 824 which diverge as they approach a distal end of sleeve 820.

Optionally, sleeve 820 is fitted with a handle 822 on its proximal side.

Sleeve 820 comprises a large lumen 826 into which a visual guiding apparatus 610 such as a hysteroscope 510 (not shown in FIG. 8) may be inserted.

Sleeve 820 comprises at least one channel 824 which turns outward (i.e. away from a central axis of sleeve 820) as it approaches a distal end of sleeve 820, such that a flexible or semi-rigid treatment probe 630 advanced through channel 824 is caused to turn outward as it extends beyond sleeve 820. Preferably, a plurality of such outward-turning channels 824 is provided (two such exemplary channels are shown in a cross-sectional view provided by FIG. 8). Thus, if a plurality of flexible or semi-rigid treatment probes 630 are advanced through a plurality of outward-turning channels 824 (such as channels 824 presented in FIG. 8), then as those treatment probes 630 extend beyond sleeve 820 they are so directed that their operating tips 633 diverge, and come to be separated by a distance larger than the diameter of sleeve 820. A plurality of operating tips 633, so directed and so oriented, if inserted into a fibroid, will be appropriately positioned to cryoablate even a large fibroid.

In a recommended mode of use, an operator inserts sleeve 820 through a cervix into a uterus, positions sleeve 820 so that its distal end is near a treatment target such as fibroid 150, and inserts a plurality of flexible or semi-rigid treatment probes 630 through a plurality of channels 824, which channels have diverging exit openings. Operating tips 633 of probes 630 are caused to penetrate a fibroid 150 as they emerge from sleeve 820, and are so oriented that they diverge as they penetrate into fibroid 150. Consequently, operating tips 633 can be positioned within fibroid 150 such that tips 633 are separated by a distance larger than the diameter of sleeve 820. In particular, operating tips 633 may thus be positioned so as to be well distributed within fibroid 150, and may be operated in cryocooling in such well-distributed positions. Thus, sleeve 820, with diverging exit openings of a plurality of channels 824, can be used to effect simultaneous and complete cryoablation of even a large fibroid by a plurality of cryoprobes cooling concurrently in a single cooling operation, without need for multi-stage cycles of cooling, thawing, repositioning of probes, and re-cooling.

Preferably, several types of sleeves 820, each with different number of channels 824 and/or with different diverging angles of channels 824, will be made available to a surgeon, who will select among them a particular configuration best suited to each particular treatment target.

Sleeve 820 may be made of metal or plastic and may be made for multiple uses, or may be designed and constructed for one-time use. Angles of divergence of channels 824 may vary within each sleeve 820, or may be uniform. Thermal sensing probes may be used in conjunction with thermal treatment probes 630, or thermal sensors may be incorporated in thermal probes 630.

It is noted that sleeves 620, 720, 820, and 920, and similar sleeves, as well as heat insulating sleeves 673 and 683 and similar heat-insulating sleeves, may be configured to accommodate treatment probes and other narrow-diameter tools only, without providing space therein for a hysteroscope or other visualization apparatus. The diameter of a hysteroscope being relatively large with respect to the diameter of thermal treatment probe such as a cryoprobe, it will for some applications be preferable to provide a relatively narrow sleeve configuration, without provision for a hysteroscope. Such a configuration enables delivery of a plurality of treatment needles to a treatment site through an elongated sleeve with individual probe channels (as shown in FIGS. 7a and 7b and in FIG. 8), or without individual probe channels (as shown in FIGS. 6a, 6b, and 6c). Such a sleeve may also be useful for delivering a plurality of diverse surgical tools, such as one or more treatment probes together with one or more thermal sensors. Such a sleeve may be steerable or have a steerable component, as shown in FIG. 5. In the case of sleeves comprising individual channels, channels may be provided for associated equipment (such as thermal sensors) as well as for treatment probes. In the case of sleeves comprising individual probe channels, such channels may be caused to diverge at the distal end of the sleeve, as shown in FIG. 8, so as to provide for an enhanced spread of treatment probes at or near the locus of treatment. Spread of treatment probes at or near a locus of treatment may also be provided by use of pre-bent treatment probes advanced through non-divergent channels, as shown in FIG. 4, and an even greater spread of treatment probes at a treatment locus may be accomplished by combining these methods, utilizing pre-bent probes advanced through divergent channels in a sleeve.

A preferred method of use of probe-delivery sleeves presented hereinabove comprises utilizing an imaging modality external to the sleeve (such as, for example, an ultrasound probe external to the sleeve and distanced therefrom) to monitor the ablation process.

An additional preferred method for using sleeves comprising individual probe channels is to provide cryoablation probes in a plurality of channels of a sleeve, insert a first ablation probe into a portion of an ablation target, cool the inserted probe to freezing temperatures, thereby fixing that probe and it's channel to a portion of an ablation target, then rotating the sleeve (if necessary) around that inserted needle until additional needles in additional channels are aligned as desired with respect to the ablation target, and there to insert additional needles into the ablation target and there operate those additional inserted needles to ablate portions of the ablation target.

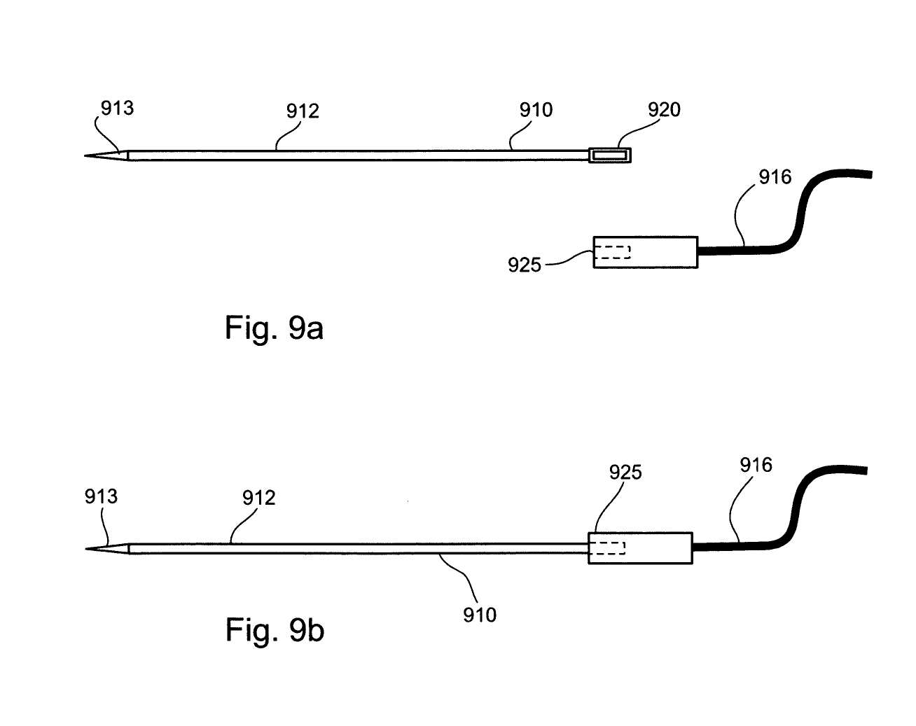

Attention is now drawn to FIGS. 9(a) and 9(b), which are simplified schematics of a treatment probe having a low-profile connector, in disconnected and connected configurations respectively, according to an embodiment of the present invention.

As has been noted in discussion of various embodiments presented hereinabove, it is often desirable to insert a thermal treatment probe into a surgical target in a body cavity under endoscopic (e.g., hysteroscopic) guidance. As discussed, it is also often desirable to remove optical instruments used for endoscopic observation during actual thermal treatment of a target, to avoid thermal damage to those optical instruments. In another aspect of treatment, it may be desired to introduce a plurality of treatment probes into a treatment target in a body cavity by sequential use of a common endoscopic tool (such as a treatment hysteroscope) to sequentially introduce a plurality of treatment probes using a single working channel (i.e. a single channel for treatment probes) within that endoscopic tool One obstacle to such procedures is found in the form of treatment probes, according to typical constructions of prior art. Treatment probes such as cryoprobes typically comprise a large-diameter connector at their proximal end, used to connect such cryoprobes to a cryogen supply. Similarly, sensors, electrical ablation probes, and other surgical tools of various sorts typically comprise proximal connectors which are wider than diameters of the probes themselves, which probes have shafts designed to fit within narrow working channels of endoscopes.

Treatment probe 910 presented in FIG. 9(a) comprises a shaft 912, a treatment head 913 operable to treat a surgical target, and what is termed herein a “low-profile” proximal connector 920. Low-profile connector 920 is provided to establish an appropriate physical connection with other objects within a treatment system. For example, if treatment probe 910 is a cryoprobe 229 designed to be cooled by Joule-Thomson cooling, then connector 920 will be designed to connect to a female connector 925 attached to a high-pressure gas supply hose 916 for supplying high-pressure cooling gas to probe 910. If treatment probe 910 is a cryoprobe 229 designed to be cooled by evaporative cooling, then connector 920 will be designed to connect to a female connector 925 attached to a source of a liquid cryogen. If treatment probe 910 is an electrical ablation probe, then connector 920 will be designed to connect to a female connector 925 attached to a source of electrical energy. If treatment probe 910 is a sensor, then connector 920 will be designed to connect to a female connector 925 connected to a data-reporting output path. In all these and similar uses, connector 920 is characterized in that its diameter is preferably inferior to, and in any case not substantially superior to, a diameter of probe 910. FIG. 9(a) shows connector 920 disconnected from female connector 925, and FIG. 9(b) shows connector 920 connected to female connector 925.

As may be seen from FIG. 9(a), disconnected probe 910 may be constructed to be substantially smooth along its entire surface, including that part of its surface which comprises connector 920. Minor exceptions to this smoothness (e.g., optional screw threads on connector 920) may be constructed in a slightly recessed manner, so as not to cause hindrance of movement of all of probe 910, including a proximal portion of probe 910 comprising connector 920, through a narrow passageway sized to the general diameter of probe 910, a passageway such as, for example, a working channel of a treatment hysteroscope or other endoscope.

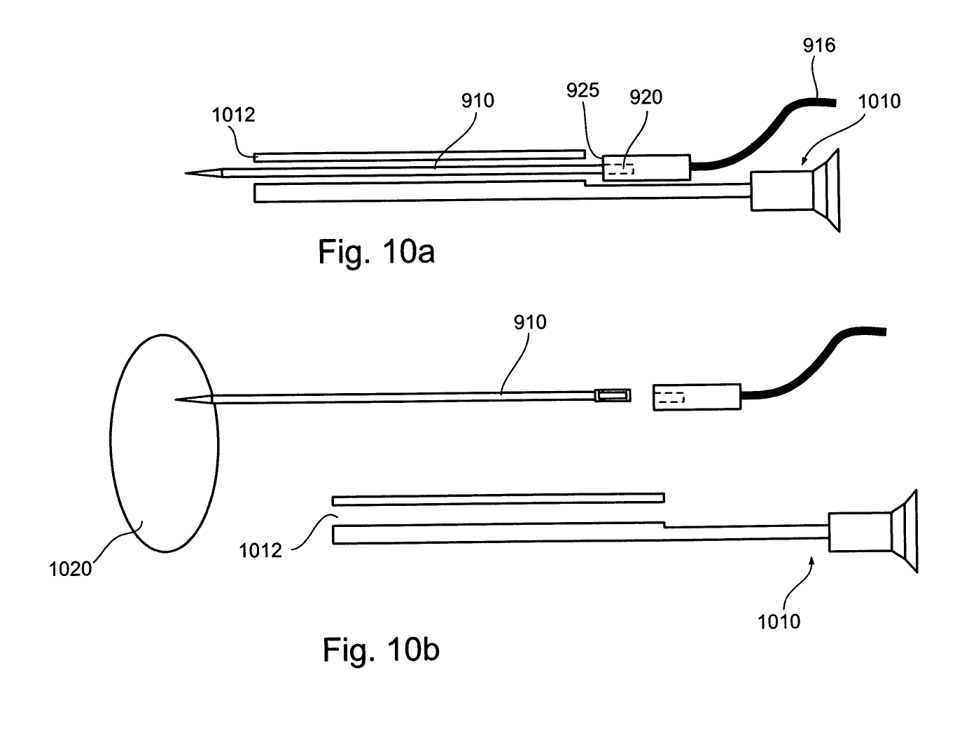

Thus, “low-profile” construction of connector 920 enables probe 910 to pass entirely through, and emerge from, a distal portion of a working channel of an endoscopic tool, as may be seen in FIG. 10.

Attention is now drawn to FIGS. 10(a) and 10(b), which present simplified schematics of a treatment-probe/endoscope combination, according to an embodiment of the present invention. FIG. 10(a) shows probe 910 inserted in a working channel 1012 of an endoscope 1010. Endoscope 1010 may be a prior-art endoscope such as those discussed hereinabove with respect to FIGS. 2(a)-2(c) of the present application, or may be one of the endoscopic (or hysteroscopic) sleeves presented above as embodiments of the present invention, or may be any other endoscopic apparatus providing a working channel for delivering a probe to a treatment target. Thus, probe 910, when connected to connector 925 as shown in FIG. 10(a), may be used in a manner similar to other probe/endoscope designs well known in the art. However, once treatment head 913 of probe 910 has been positioned appropriately with respect to a treatment target and preferably fixed to that target, connector 925 may be disconnected from connector 920, allowing endoscope 1010 to be partially or entirely removed from the target vicinity, with endoscope 1010 being withdrawn and connector 920 passing unhindered through working channel 1012 of endoscope 1010 as shown in FIG. 10(b), after which connector 925 may be reconnected to connector 920 of probe 910, enabling probe 910 to treat the target.

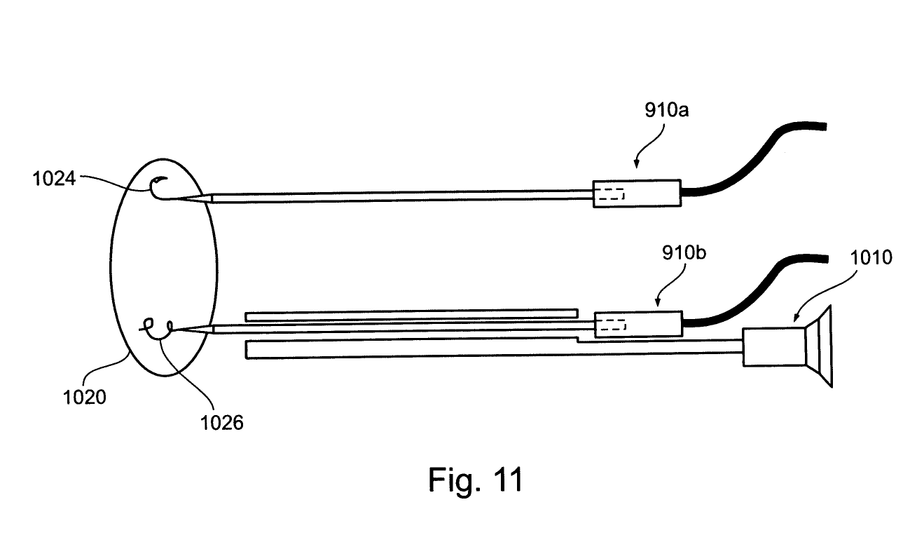

Attention is now drawn to FIG. 11, which is a simplified schematic demonstrating use of low-profile connector 920 to enable use of an endoscope with a single working channel to position a plurality of treatment probes 910 into a target for treatment of that target, according to an embodiment of the present invention. As shown in the preceding paragraph, a treatment probe 910 having low-profile connector 920 may be positioned in a treatment target by use of endoscope 1010, after which endoscope 1010 may be removed leaving probe 910 in place. Repetition of this procedure enables placement of a plurality of probes 910 in a common target 1020, as shown in FIG. 11. (Optionally, the last probe to be inserted may be a probe without low-profile connector 920. Such a probe would of course remain within working channel 1012 during treatment of target 1020.)

In a preferred procedure, each probe 910 is fixed to target 1020 before removal of endoscope 1010 from the vicinity of target 1020. If probe 910 is a cryoprobe, it is recommended to operate probe 910 briefly, or at a low power setting, causing freezing of tissues adjacent to treatment head 913 of probe 910 and consequent adherence of those tissues to treatment head 913, prior to removal of endoscope 1010. Alternatively, probe 910 may be provided with an attaching element such as a “fishhook” appendage 1024, or a “corkscrew” appendage 1026, by means of which secure positioning of probe 910 with respect to target 1020 may be assured prior to removal of endoscope 1010.

Low-profile connectors 920 may be fitted to a variety of treatment probes and various tools sized to be introduced through working channel 1012. Thus, cryoprobes of different cooling powers, or combinations of treatment probes and sensing probes, or combinations of cooling probes and heating probes used for protection of vital organs near a treated organ, may be thus equipped with low-profile connectors 920 and sequentially introduced through a same working channel 1012. Once all desired treatment probes have been thus introduced, endoscope 1010 is optionally removed and may be replaced by other tools, such as for example an imaging device of a different type, an ultrasound probe for example.

Attention is now drawn to FIGS. 12(a) and 12(b), which present simplified schematics of a side view and of a cross-sectional view respectively of a treatment probe insertion and manipulation apparatus, according to an embodiment of the present invention.

As presented in FIGS. 12(a) and 12(b), treatment probe insertion and manipulation apparatus 1200 comprises a sleeve 1210 having a lumen 1212 and a working channel 1214. Lumen 1212 is large enough to accommodate a scope 610 (e.g. a hysteroscope). Apparatus 1200 further comprises a manipulator 1220, sized to fit within working channel 1214. Manipulator 1220 comprises a channel 1222 sufficiently large to accommodate a treatment probe 1216, which may be a cryoprobe or other probe.

Thus, treatment probe 1216 is insertable in channel 1222 of manipulator 1220, and manipulator 1220 is insertable in working channel 1214 of sleeve 1210.

Preferably, channel 1222 has a curve 1226 at distal end 1224 of manipulator 1220, such that tip 1230 of probe 1216 curves away from the axis of manipulator 1220 as probe 1216 is pushed forward through channel 1222.

A handle 1229 on manipulator 1220 allows rotation of manipulator 1220 within working channel 1222, thus enabling to control the angled direction at which probe 1216 exits distal end 1224 of manipulator 1220. Thus, with probe 1216 inserted in manipulator 1220, manipulator 1220 inserted in sleeve 1210, and sleeve 1210 inserted in a patient, an operator may use handle 1229 to control the direction in which probe 1216 extends from manipulator 1220 and from sleeve 1210, without needing to rotate sleeve 1210 and without needing to rotate scope 610, thus providing enhanced control and enhanced convenience during treatment of a therapeutic target.

A gas connector 1219 or other connector or handle provided on a distal portion of probe 1216 may be used for pushing probe 1216 into channel 1222.

Alternatively, manipulator 1220 may be replaced with the steerable delivery guide such as has been described hereinabove in particular with respect to FIG. 5.

Sleeve 1210 may be a standard sleeve used for hysteroscopy. One might, for example, use a 1.5 to 2.5 mm treatment probe together with a manipulator 2.5 to 5 mm diameter and an endoscope 3 to 6 mm in diameter, these combined and positioned appropriately so as to be useable in a sleeve 1210 whose outside diameter does not exceed 9 mm. For example, one might use a 1.5 mm treatment probe in a 2.5 mm manipulator and a 6 mm scope, or a 2.5 mm probe in a 5 mm manipulator and a 3.5 mm scope, or some similar combination.

FIG. 12(b) presents a cross-sectional view of apparatus 1200, showing with clarity the spatial relationship of the various components described above. In this drawing, scope 610 is asymmetrically positioned within sleeve 1210.

As seen in the Figure, sleeve 1210 optionally comprises a secondary channel 1299 which may be used for flushing with liquid the vicinity of a treatment target, for gas inflation of a body cavity, for insertion of an additional manipulator 1220, or for similar surgical uses.

Optionally, lumen 1212 and endoscope 650 may be positioned axially in sleeve 1210, permitting a symmetrically or nearly symmetrical construction of sleeve 1210. For example, a 3 mm scope centrally situated in a 9 mm sleeve will leave room for a plurality of 2.5 mm manipulators around it.

Attention is now drawn to FIG. 13(a), which is a simplified schematic of a heat insulator useful for cryosurgery, according to an embodiment of the present invention.

Heat insulator 1300 presented in FIG. 13(a) may be seen as a preferred embodiment of heat-insulating sleeve 673 which was discussed hereinabove in the context of system 600 and presented in FIGS. 6(f)-6(h). Insulator 1300 is useful to protect a cervix during thermal treatment of a uterus.

Heat insulator 1300 comprises a body 1372 made of heat-insulating material, which may be flexible materials or firm materials.

Insulator 1300 comprises a lumen 1374 sized to accommodate one or more cryoprobe shafts or cryogen supply and exhaust lines used to operate one or more cryoprobes. In operation, cryoprobe shafts or cryogen supply/exhaust lines are slideably fitted inside lumen 1374 of insulator 1300.

Distal end 1371 of body 1372 is preferably narrow to permit easy penetration of a cervix, and is rounded so as not to cause injury to a cervix into which insulator 1300 is introduced.

Proximal end 1379 of body 1372 is preferably configured for easy holding by an operator, thereby facilitating the task of pushing insulator 1300 into position for protecting a cervix during cryoablation.

In a preferred embodiment, a stop 1376 prevents heat insulator 1300 from being pushed pass the cervix.

An optional bulge 1378 is provided. Bulge 1378 may be pushed passed the muscular cervix, thereby stabilizing insulator 1300 in place within the cervix opening by slightly impeding withdrawal of insulator 1300 from the cervix.

Thus, insulator 1300 is shaped and dimensioned so as to penetrate a dilated cervix and to remain therein, and to contain cold shafts and/or cryogen lines of cryoprobes used within a uterus, thereby easily and conveniently protecting the cervix from damage by cold cryoprobe shafts and cryogen lines passing through the cervix during thermal treatment of a uterus.

Attention is now drawn to FIG. 13(b), which presents a simplified schematic of a cross-sectional view of a heat insulator inserted into a cervix opening, according to an additional embodiment of the present invention.

FIG. 13(b) presents a heat insulator 1310 inserted into a cervix opening 690. Insulator 1310 comprises a body 1382 preferably made of flexible material having poor hear conductivity, such as, for example, silicon rubber.

Heat insulator 1310 is similar in shape and in function to heat insulator 1300, yet differs therefrom in that insulator 1310 comprises a slot 1381 cut along the full length of heat insulator body 1382, so that lumen 1384 of insulator 1310 is accessible from beside insulator 1310. Thus, when it is desired to insert cryoprobe shafts or other coolable objects into lumen 1384 to protect a cervix from exposure thereto, such shafts or other objects can be pushed through slot 1381 into 1384 from the side, providing insulator 1310 with enhanced convenience of use. FIG. 13(b) presents three shafts so inserted, labeled 1389a, 1389b and 1389c in this Figure.

As described above with respect to insulator 1300, insulator 1310 similarly preferably comprises a stop to prevent insulator 1310 from being pushed past the cervix, and a bulge which may be pushed passed the muscular cervix to stabilize insulator 1310 in place within the cervix opening.

Attention is now drawn to FIG. 13(c), which presents a simplified schematic of a cross-sectional view of a heat insulator 1320 of split-body construction inserted into a cervix opening, according to yet another embodiment of the present invention.

Heat insulator 1320 presented in FIG. 13(c) is similar to insulator 1300 discussed hereinabove, and differs therefrom in that insulator 1320 comprises a body divided into two semi-independent parts labeled 1392a and 1392b in FIG. 13(c).

Body parts 1392a and 1392b of split heat insulator 1320 are preferably made of materials having poor hear conductivity, such as plastic.

As discussed above with respect to insulators 1300 and 1310, in operation cryoprobe shafts or gas lines are fitted inside lumen 1384 of heat insulator 1320, and are thereby prevented from contact with cervix 690 during thermal treatment of the uterus. FIG. 13(c) shows such shafts, labeled 1389a, 1389b and 1389c positioned within lumen 1384 of insulator 1320, with insulator 1320 closed and positioned within a cervix 690.

Body parts 1392a and 1392b are independent or partially independent. That is, they may be completely separable, or may be joined along some line of contact with a hinge-like structure. In either case, body parts 1392a and 1392b may be opened and separated sufficiently to allow shafts 1389 to be so positioned that when parts 1392a and 1392b are joined, a lumen 1384 is formed and contains shafts 1392, and joined parts 1392 serve to isolate shafts 1392 from tissues surrounding insulator 1320.

Parts 1392a and 1392b may be joined using a “dovetail” construction, or by means of a clasp at proximal end 1379 of insulator 1320, (which clasp preferably does not penetrate cervix 690), or by any similar means of joining.

Thus, heat insulator 1320 is similar in shape and in function to heat insulator 1300, yet differs therefrom in respect of its split two-part body construction. Thus, when it is desired to insert cryoprobe shafts or other coolable objects into lumen 1384 of insulator 1320 to protect a cervix from exposure thereto, insulator 1300 can be opened, separating or partially separating parts 1392a and 1392b, such shafts or other objects can inserted between those parts, and parts 1292a and 1392b can then be closed and/or connected to each other to form a closed insulator 1320, which is then operable to protect a cervix during cryoablation in a uterus.

As described above with respect to insulator 1300, insulator 1320 similarly preferably comprises a stop to prevent insulator 1320 from being pushed past the cervix, and a bulge which may be pushed passed the muscular cervix to stabilize insulator 1320 in place within the cervix opening.

Thus, when un-slotted heat insulator 1300 is used, for example, with the embodiment presented in FIG. 6, a probe or probes are inserted through lumen 1374 before the probe is inserted into the patient. In contrast, slotted heat insulator 1310 or split heat insulator 1320 may be positioned around probe shafts after those probes are already inserted into a patient.

Heat insulators 1300, 1310 and 1320 may be used with the various probes and probe delivery systems disclosed hereinabove, and in particular with devices presented in FIGS. 6, 9, 10 and 11.

It should be appreciated that the invention presented hereinabove may be used as described, or with minor and obvious alterations, to treat lesions other than uterine fibroids. The invention may be used in body cavities other than the uterus, and may be used to treat organs other than the uterus, and to treat treatment targets other than fibroids.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for delivering a treatment probe to a treatment target within a body cavity, comprising a delivery sleeve having a distal portion operable to be inserted into a body cavity, said delivery sleeve being sized to accommodate at least one treatment probe and being operable to deliver a treatment head of said treatment probe to a vicinity of said treatment target when said distal portion of said sleeve is inserted in said body cavity, said delivery sleeve being further characterized in that said sleeve comprises an opening running along its length, said opening being sized to permit passage therethrough of said treatment probe.

2. The system of claim 1, further comprising a treatment probe lumen sized to accommodate said at least one treatment probe, said lumen being switchable between an open state permitting a treatment probe to enter and exit said treatment probe lumen and a closed state which prevents treatment probes from entering and from exiting said treatment probe lumen.

3. The system of claim 2, wherein said open state is characterized by a first configuration wherein said opening is aligned with said treatment probe lumen, enabling translation of a treatment probe into and out of said lumen, and said closed state is characterized by a second configuration wherein said opening is unaligned with said treatment probe lumen and translation of a treatment probe into and out of said treatment probe lumen is prevented.

4. The system of claim 3, wherein transition from said open state to said closed state may be effected by rotating a cover of said delivery sleeve with respect to a body of said delivery sleeve.

5. The system of claim 1, wherein said delivery sleeve is sized to accommodate both said at least one a treatment probe and also a visual guiding apparatus.

6. The system of claim 5, wherein said treatment probe is prevented from passage through said opening when said probe and said visual guiding apparatus are both inserted in said sleeve, and said treatment probe is enabled to pass through said opening when said probe is inserted in said sleeve and said visual guiding apparatus is not inserted therein.

7. The system of claim 5, wherein said visual guiding apparatus is an optical hysteroscope.

8. The system of claim 1, further comprising a plurality of treatment probes.

9. The system of claim 1, wherein said distal portion of said delivery sleeve is so shaped and dimensioned as to be capable of insertion into a uterine lumen through a cervix.

10. The system of claim 1, wherein said treatment probe is a cryoprobe.

11. The system of claim 1, wherein said delivery sleeve comprises an echogenic surface.

12. The system of claim 1, wherein at least a portion of said delivery sleeve comprises heat-insulating material.

13. The system of claim 1, further comprising a second sleeve, which second sleeve comprises heat-insulating material.

14. The system of claim 13, further comprising a third sleeve, which third sleeve comprises heat-insulating material.

15. A system for delivering a plurality of thermal treatment probes to a treatment target within a body cavity, comprising:
(a) a sleeve having a first lumen sized to accommodate an optical hysteroscope; and
(b) a plurality of working channels each sized to accommodate a treatment probe.

16. The system of claim 15, wherein said first lumen is positioned centrally within said sleeve, and said working channels are positioned circumferentially around said central lumen.

17. The system of claim 15, wherein said working channels are positioned asymmetrically with respect to said first lumen.

18. The system of claim 15, further comprising a plurality of treatment probes each sized to be insertable within at least one of said working channels.

19. The system of claim 18, wherein at least one of said treatment probes comprises a proximal connector operable to connect said at least one probe to a cryogen source, said connector being of a diameter not substantially greater than a diameter of said probe.

20. The system of claim 18, wherein at least one of said treatment probes is a cryoprobe.

21. The system of claim 18, wherein at least one of said treatment probes is a pre-bent treatment probe.

22. The system of claim 21, further comprising a plurality of pre-bent treatment probes disposable within said plurality of working channels in such orientation that when said pre-bent treatment probes extend from a distal end of said sleeve, a distance of one of said treatment heads from at least one other of said treatment heads is greater than a diameter of said sleeve.

23. The system of claim 15, wherein distal ends of at least some of said plurality of channels diverge as they approach a distal end of said sleeve.

24. A system for delivering a thermal treatment probe to a treatment target within a body cavity, comprising:
(a) a sleeve having a first lumen sized to accommodate a visual guiding apparatus;
(b) a working channel sized to accommodate a treatment probe; and
(c) a treatment probe which comprises a proximal connector operable to connect said probe to a cryogen source, said connector being of a diameter not substantially greater than a diameter of said probe.

25. The system of claim 24, wherein said treatment probe is a cryoprobe.

26. The system of claim 24 wherein said treatment probe is a pre-bent probe.

27. A probe sized and shaped to traverse a working channel of an endoscope, comprising:
(a) a shaft having a maximum diameter D sized and shaped to enable passage through a working channel of an endoscope;
(b) a treatment head positioned at a distal portion of said shaft and operable to treat a target tissue within a body cavity when supplied with a material substance transported to said treatment head through said shaft; and
(c) a connector positioned at a proximal portion of said shaft and operable to connect said shaft to a source of said material substance, said connector having a diameter not superior to said diameter D of said shaft.

28. The probe of claim 27, wherein said treatment head comprises a member operable to be anchored to said target tissue.

29. The probe of claim 27 wherein said treatment head is operable to be cooled when a cryogen is supplied through said connector.

30. An insulating device for protecting a cervix during thermal cryoablation within a uterus, comprising
(a) a distal portion formed and dimensioned so as to be operable to non-destructively penetrate a cervix; and
(b) a lumen sized to accommodate cryogen supply and exhaust lines of at least one cryoprobe,
said device comprising heat-insulating materials operable to at least partially protect tissues of a cervix from thermal damage when said device is positioned within said cervix and a cryoprobe having cryogen supply and exhaust lines passing within said lumen is used to thermally ablate tissues within said uterus.

31. The device of claim 30, further comprising a proximal portion sufficiently broad to prevent penetration of said proximal portion through said cervix.

32. The device of claim 31, further comprising a bulge in a region of said distal portion of said device, said bulge being so positioned as to impede withdrawal of said device from said cervix once said distal portion of said device is inserted into said cervix.

33. The device of claim 30, further comprising a longitudinal slit enabling to push a cryogen supply line into said lumen of said device from a position alongside said device.

34. The device of claim 30, wherein said lumen is openable and closeable, opening of said device enabling lateral introduction of an adjacent cryogen supply line into said lumen, and closing of said device enabling to protect cervical tissues when said device is inserted in said cervix and a cryogen supply line introduced into said lumen is cooled.

35. A method for delivering a plurality of treatment probe heads to a treatment target within a body cavity, comprising:
(a) providing a visual guiding apparatus, a first treatment probe having a first treatment head operable to treat said treatment target, a second treatment probe having a second treatment head operable to treat said treatment target, and a sleeve configured to accommodate said visual guiding apparatus and at least one of said first and second treatment probes, said sleeve having an opening running along its length and having a distal portion insertable into a body cavity;
(b) utilizing said visual guiding apparatus to guide placement of said first treatment head of said first treatment probe at a position appropriate for treating said treatment target;
(c) freeing said first treatment probe from said sleeve;
(d) inserting said second treatment probe into said sleeve; and
(e) utilizing said visual guiding apparatus to guide placement of said second treatment head at a position appropriate for treating said treatment target.

36. A method for delivering a plurality of treatment probe heads to a treatment target within a body cavity, comprising:
(a) providing a visual guiding apparatus, a plurality of treatment probes each having a treatment head operable to treat said treatment target, and a sleeve sized to accommodate said visual guiding apparatus and at least one of said plurality of treatment probes, said sleeve having an opening running along it's length and having a distal portion insertable into a body cavity;
(b) inserting said visual guiding apparatus and a first treatment probe into said sleeve and inserting said distal portion of said sleeve into said body cavity;
(c) utilizing said visual guiding apparatus to guide placement of a treatment head of said first treatment probe at a position appropriate for treating said treatment target;
(d) removing said visual guiding apparatus from within said sleeve;
(e) displacing said sleeve so as to free said first treatment probe from containment within said sleeve;
(f) reinserting said visual guiding apparatus into said sleeve and into said body cavity;
(g) inserting a second treatment probe into said sleeve and into said body cavity; and
(h) utilizing said visual guiding apparatus to guide placement of said second treatment probe at a position appropriate for treating said treatment target.

37. The method of claim 36, wherein at least one of said first and second treatment probes is a cryoprobe.

38. The method of claim 37, further comprising cooling said first treatment probe, and thereby causing said first treatment probe to adhere to said treatment target, before displacing said sleeve to free said first treatment probe from said sleeve.

39. The method of claim 36, further comprising attaching said treatment head of said first treatment probe to said target before removing said visual guiding apparatus from within said body cavity.

40. The method of claim 36, wherein said sleeve is at least partially constructed of heat-insulating material.

41. The method of claim 40, further comprising positioning within a cervix a portion of said sleeve, which portion comprises heat-insulating material, and maintaining said portion positioned within said cervix while using a treatment probe to cool a treatment target within a uterus.

42. The method of claim 36 further comprising utilizing said sleeve to introduce a heat source into a body cavity, and using said heat source to heat first tissues within said body cavity while utilizing said cryoprobe to cool second tissues within said body cavity.

43. The method of claim 42, where said heat source is a warm-water balloon.

44. The method of claim 36, further comprising inserting at least one cryogen supply tube attached to at least one treatment probe through a heat-insulating sleeve prior to insertion of said treatment probe into said treatment target, and positioning said heat-insulating sleeve within a cervix prior to thermal operation of said treatment probe.

45. The method of claim 36, further comprising installing a heat-insulating sleeve over at least one cryogen supply tube attached to at least one treatment probe, and positioning said heat-insulating sleeve within a cervix prior to thermal operation of said treatment probe.

46. A method for inserting multiple treatment probes into a target tissue within a body cavity, comprising the steps of:
(a) supplying a first treatment probe which comprises:
(i) a shaft having a maximum diameter D, said shaft being sized and shaped to enable passage of said treatment probe through a working channel of an endoscope;
(ii) a treatment head connected to a distal portion of said shaft and operable to treat a target tissue within a body cavity when supplied with a material substance transported to said treatment head through said shaft; and
(iii) a connector positioned at a proximal portion of said shaft and operable to connect said shaft to a source of said material substance, said connector having a diameter not substantially superior to said diameter D of said shaft;
(b) inserting said first treatment probe into working channel of an endoscope;
(c) inserting said endoscope into said body cavity and positioning said treatment head of said first treatment probe in a vicinity of said target tissue;
(d) disconnecting said connector from said source of material substance, if connected;
(e) retracting said endoscope from said body cavity while leaving said treatment head of said first probe positioned at said target tissue, said shaft of said first probe transiting said working channel of said endoscope and exiting from a distal end of said working channel as said endoscope is retracted;
(f) inserting a second treatment probe into said working channel of said endoscope and re-inserting said endoscope into said body cavity; and
(h) positioning a distal portion of said second treatment probe in a vicinity of said target tissue.

47. The method of claim 46 and further comprises the step of anchoring said first treatment probe to said target tissue prior to retracting said endoscope from said body cavity.

48. The method of claim 47, further comprising wherein anchoring said first treatment probe to said target tissue by cooling a portion of said probe adjacent to said target tissue, thereby causing said tissue to freeze and to adhere to said probe.

49. The method of claim 46 wherein at least one of said first and second probes is a cryoprobe.

50. The method of claim 46 wherein said endoscope is a hysteroscope.

51. The method of claim 46 wherein said endoscope comprises a single working channel.

52. The method of claim 46 wherein said endoscope comprises a sleeve having a lumen for a visual guiding apparatus and a plurality of working channels sized to accommodate treatment probes.
